(19) United States
(12) Patent Application Publication
(10) Pub. No.: US 2024/0391941 A1
(43) Pub. Date: Nov. 28, 2024

(54) NAPHTHYRIDINONE DERIVATIVES FOR THE TREATMENT OF A DISEASE OR DISORDER

(71) Applicant: Novartis AG, Basel (CH)

(72) Inventors: Atwood CHEUNG, Arlington, MA (US); Donglei LIU, Dover, MA (US); Stefan PEUKERT, Arlington, MA (US); John TRAUGER, Cambridge, MA (US)

(21) Appl. No.: 18/670,859

(22) Filed: May 22, 2024

Related U.S. Application Data
(60) Provisional application No. 63/504,099, filed on May 24, 2023.

(51) Int. Cl. C07F 9/6561; A61K 31/4375; A61K 45/06; C07D 471/04

(52) U.S. Cl. CPC C07F 9/6561; A61K 31/4375; A61K 45/06; C07D 471/04

(57) ABSTRACT

The present disclosure relates to a compound which is a phosphoric acid ester derivative of a compound of formula (I):

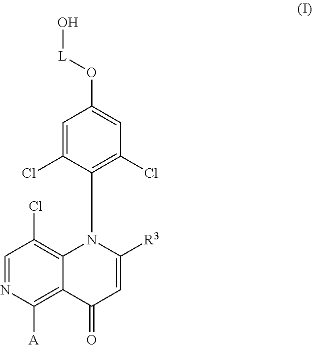

wherein A, L and R³ are as described herein, as well as compositions and methods of using such compound.

### RELATED APPLICATION

This application claims the benefit of and priority to U.S. Provisional Application No. 63/504,099, filed May 24, 2023, the entire contents of which are hereby incorporated by reference in their entirety.

### TECHNICAL FIELD

The present disclosure is directed to naphthyridinone compounds, the use thereof as pro-drugs for inhibiting the GIRK1/4 channel and methods of treating a disease or disorder using the same.

### BACKGROUND

A normal cardiac cycle begins in the sino-atrial node, which produces an excitatory electrical stimulus that propagates in an orderly fashion throughout the atrial and ventricular myocardium to induce a contraction (systole). At the cellular level, the excitatory electrical impulse triggers the cardiac action potential. This is characterized by an initial, rapid membrane depolarization followed by a plateau phase and subsequent repolarization to return to resting membrane potential. The cardiac action potential govems signal propagation throughout the heart. For example, the rate of initial cellular depolarization determines the velocity at which excitatory stimuli propagate. The duration of the repolarization phase determines the action potential duration (APD) and the refractory period, or time in which a cardiomyocyte cannot respond to another electrical stimulus.

Abnormalities in the cardiac action potential are associated with arrhythmia. For example, excessive reduction of action potential duration and the associated refractory period can provide a substrate for so-called re-entrant tachyarrhythmia. In this condition. instead of propagating normally, a cardiac impulse feeds back upon itself via excitable tissue to form a re-entrant circuit (Waldo et al., Lancet 347, 1189-1193). Existing class III anti-arrhythmic drugs are thought to work by lengthening the APD and associated effective refractory period (ERP), thereby minimizing the risk of re-excitation and subsequent formation of fibrillatory re-entry circuits (Singh et al., British Journal of pharmacology 39, 675-687).

Certain class III anti-arrhythmic drugs (e.g., sotalol) are used in the treatment of atrial fibrillation (AF). AF is the most common form of sustained cardiac arrhythmia in humans and is characterized by fibrillatory contractions that compromise atrial function. AF is associated with adverse cardiovascular events. In particular, the presence of AF is an independent risk factor for thromboembolic stroke, heart failure and all-cause mortality (Estes et al., (2008). Journal of the American College of Cardiology 51, 865-884) (Fang et al., 2008. Journal of the American College of Cardiology 51, 810-815). AF can also reduce quality of life in some patients by inducing palpitations and reducing exercise tolerance (Thrall et al., 2006. The American journal of medicine 119, 448.e441-419). The goal of anti-arrhythmic therapy for AF is to avoid these adverse effects and outcomes.

A drawback of existing Class III anti-arrhythmic drugs is that they act to prolong effective refractory period in both atria and ventricles. Excessive prolongation in ventricular tissue lengthens QTc interval and can be pro-arrhythmic. and certain drugs with this mechanism of action (e.g., dofetilide) are known to induce potentially life-threatening ventricular arrhythmias such as Torsades de Pointes (Redfern et al., 2003. Cardiovascular Research 58, 32-45). There is thus a need for a novel anti-arrhythmic therapy for AF that targets atrial, and not ventricular, tissue selectively.

The configuration and duration of the cardiac action potential is controlled at the cellular level by the action of multiple different transmembrane ion channels. For example, the initial depolarization phase is mediated by influx of sodium ions via the cardiac-specific Naᵥ1.5 channel. Potassium channels are responsible for the latter phase of repolarization, and thus help regulate the overall duration of the action potential. Indeed, class III anti-arrhythmic drugs that target potassium channels (e.g., dofetilide) prolong both action potential duration and effective refractory period. There are several different varieties of transmembrane potassium channel (Schmitt et al., 2014 Physiological reviews 94, 609-653; Tamargo et al., 2004. Cardiovascular research 62, 9-33), including:
Voltage-gated channels (Kv1-9)
Calcium-activated channels (KCal-2)
Tandem pore domain channels (e.g., TASK)
Inwardly rectifying channels (Kirl-6)

While most cardiac potassium channels contribute to repolarization in both atrial and ventricular tissues in humans, two—Kᵥ1.5 and GIRK1/4 (i.e., G-protein regulated inwardly rectifying potassium channel 1/4)—are thought to be expressed solely in atria (Gaborit et al., 2007. The Journal of physiology 582, 675-693). This atrial-specific pattern of expression makes these particularly attractive targets for novel anti-arrhythmic therapies for AF, as they should not have the adverse ventricular effects of existing Class III drugs such as dofetilide.

Mammals express four different GIRK channels (GIRK 1, 2, 3 and 4; encoded by KCNJ3, KCNJ6, KCNJ9 and KCNJ5, respectively). These transmembrane spanning proteins are arranged as tetramers (either homo or heterotetramers) to form a functional potassium channel (Krapivinsky et al., 1995. Nature 374, 135-141). These channels are ligand-gated (i.e., regulated by binding of ligands to Gi-protein coupled receptors present in the same cell membrane). For example, the GIRK 1/4 channel is a heterotetramer (two subunits each of GIRK1 and GIRK4) expressed strongly in sino-atrial and atrioventricular nodes as well as the atrial myocardium (Wickman et al., 1999. Annals of the New York Academy of Sciences 868, 386-398). One function of this channel is to mediate autonomic regulation of heart rate (HR). Acetylcholine released upon parasympathetic stimulation of cardiac vagal efferent neurons binds to Gi-coupled M2 muscarinic receptors in heart. This liberates Gβγ subunits, which in turn open GIRK1/4 channels to permit efflux of potassium from cardiomyocytes and so promote membrane repolarization. In the spontaneously depolarizing pacemaking cells of the sino-atrial node, the magnitude of this repolarization dictates the timing between depolarizations, and hence heart rate. Because it is regulated by acetylcholine, the current mediated by GIRK1/4 channels is called I_{KAch} (Wickman et al., 1999).

Several lines of evidence point toward GIRKI/4 as a desirable anti-arrhythmia target for AF. In animals, vagal nerve stimulation promotes acetylcholine release from vagal afferents and an increase in I_{KAch}. This in turn shortens atrial (but not ventricular) action potential duration and effective refractory period and can induce AF via a re-entry mechanism (Hashimoto et al., 2006. Pharmacological research: the official journal of the Italian Pharmacological Society 54, 136-141). In atrial tissues from humans with persistent AF as well as from animals subjected to atrial rapid pacing (an accepted model for promoting electrical remodeling and susceptibility to AF), I_{KAch} has been shown to be dysregulated. Specifically, the channel tends to be constitutively open, even in the absence of acetylcholine (Cha et al., 2006. Circulation//3. 1730-1737; Voigt et al., 2014. Advances in pharmacology (San Diego, Calif) 70, 393-409). In these studies, it is observed in patients and animals that atrial APD/ERP is short. Thus, the development of GIRK1/4 blockers would be beneficial in the treatment of a range of cardiac-related diseases.

Pro-drugs have emerged as valuable tools in medicinal chemistry to e.g., enhance the aqueous solubility and crystallinity of drug molecules. These pro-drugs are designed to undergo enzymatic or chemical hydrolysis to release the active drug after administration, thereby improving its pharmaceutical properties.

For these reasons, there remains a need for small molecule pro-drugs which convert in vivo to inhibitors of GIRK1/4.

### SUMMARY

In a first aspect, disclosed herein is a compound which is a phosphoric acid ester derivative of a compound of formula (I):

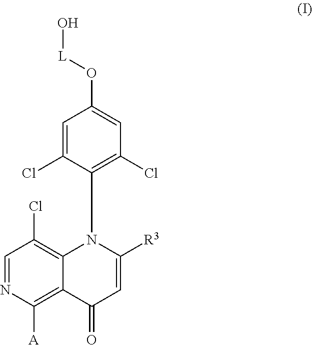

or a pharmaceutically acceptable salt thereof, wherein
L is (C₁-C₆)alkylene optionally substituted with one or more —OH;
A is —OR² or (C₁-C₆)alkyl optionally substituted with one or more substituents independently selected from —SO₂(C₁-C₄)alkyl, —NHC(O)R^{a}, and —C(O)NHR^{b};
R² is (C₁-C₆)alkyl substituted with one or more substituents independently selected from —NHC(O)R^{c} and —C(O)NHR^{d}, wherein the (C₁-C₆)alkyl is optionally further substituted with one or more substituents independently selected from halogen, —OH and —CN;
R^{a}, R^{b}, R^{c}, and R^{d} are each independently selected from H and (C₁-C₆) alkyl optionally substituted with one or more —OH; and
R³ is (C₁-C₄)alkyl.

In another aspect, the present disclosure relates to a crystalline form of the compound of the present disclosure, or a pharmaceutically acceptable salt thereof, as defined herein.

In another aspect, the present disclosure relates to a pharmaceutical composition comprising the compound of the present disclosure, or a pharmaceutically acceptable salt thereof, as defined herein and one or more pharmaceutically acceptable carriers.

In another aspect, the present disclosure relates to a combination comprising the compound of the present disclosure, or a pharmaceutically acceptable salt thereof, as defined herein and one or more pharmaceutical agents.

In another aspect, the present disclosure relates to a method for treating a disease or disorder comprising administering to a patient in need thereof a therapeutically effective amount of the compound of the present disclosure, or a pharmaceutically acceptable salt thereof, as defined herein.

In some embodiments, the disease or disorder is selected from cardiac arrhythmia, atrial fibrillation, bradyarrhythmia, bradycardia, heart block, sick sinus syndrome, parasympathetic hyperactivation, primary hyperaldosteronism, hypotension, and vasovagal syncope. In some embodiments, the disease or disorder is responsive to the inhibition of the GIRK1/4 receptor.

In another aspect, the present disclosure relates to a method for treating a disease or disorder comprising administering to a patient in need thereof a therapeutically effective amount of the compound of the present disclosure, or a pharmaceutically acceptable salt thereof, as defined herein.

In yet another aspect, the present disclosure relates to a method for maintaining a sinus rhythm after cardioversion in a patient with persistent or recent onset of atrial fibrillation or preventing a recurrence in a patient with paroxysmal atrial fibrillation comprising administering to a patient in need thereof the compound of the present disclosure, or a pharmaceutically acceptable salt thereof, as defined berein.

In another aspect, the present disclosure relates to the compound of the present disclosure, or a pharmaceutically acceptable salt thereof, as defined herein for use as a medicament.

In another aspect, the present disclosure relates to the compound of the present disclosure, or a pharmaceutically acceptable salt thereof, as defined herein for use in the treatment of a disease or disorder.

In yet another aspect, the present disclosure relates to the compound of the present disclosure, or a pharmaceutically acceptable salt thereof, as defined herein for use in the manufacture of a medicament for treating a disease or disorder.

In still another aspect, the present disclosure relates to use of the compound of the present disclosure, or a pharmaceutically acceptable salt thereof, as defined herein in the treatment of a disease or disorder.

Other features and advantages of the disclosure will be apparent from the following detailed description and claims.

### BRIEF DESCRIPTION OF THE DRAWINGS

FIG. 1 is a plot showing the effect of the parent compound of the compound of Example 1 on AERP in an anesthetized guinea pig model. AERP (milliseconds) was measured at the baseline and at the basal level induced by fingolimod (0.1 mg/kg). In this example, the parent compound of the compound of Example I was administered by intravenous infusion for 60 minutes, equating to a total dose of 3 mg/kg. As AERP was most stable in the last 30 minutes of the infusion, PK/PD relationships were assessed during this time period. Control treatments included the parent compound of the compound of Example 1 alone (saline+C-13), fingolimod alone and no added treatments (neither fingolimod nor the parent compound of the compound of Example 1).

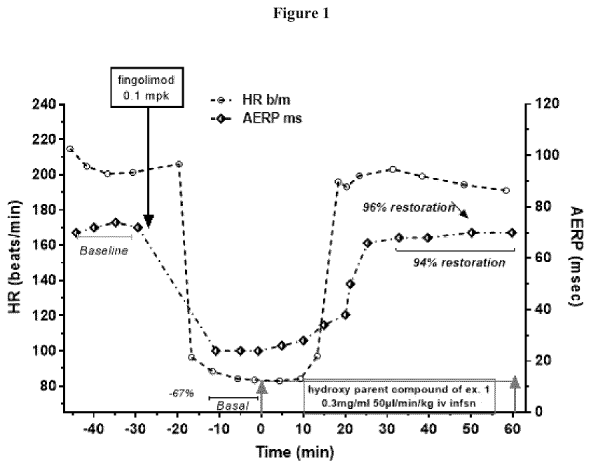

FIG. 2 is a plot showing the PK/PD relationship in guinea pig model for AERP restoration. Values represent all individual data points for percent AERP restoration and the corresponding total plasma level of the parent compound of the compound of Example 1 during the steady state period 30-60 minutes after the start of infusion across all doses and experiments.

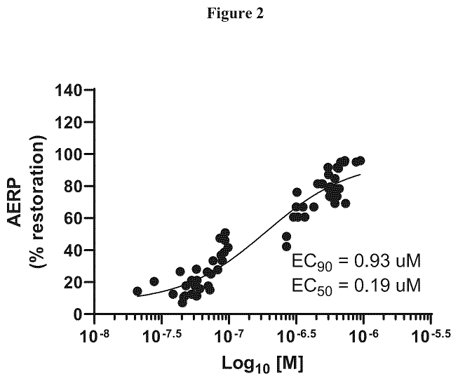

### DETAILED DESCRIPTION

In certain aspects, the present disclosure provides substituted naphthyridinone compounds. and pharmaceutical compositions thereof. In particular, such substituted compounds are useful as pro-drugs which convert in vivo to inhibitors of the GIRK1/4 receptor and have good oral bioavailability and thus can be used to treat or prevent a disease or condition.

Additionally, it has been surprisingly and unexpectedly found that the compound of the present disclosure, or a pharmaceutically acceptable salt thereof, is advantageously obtained in a stable form while being suitable for use as pro-drug of the compound of formula (I). It has been found that the compound of the present disclosure, or a pharmaceutically acceptable salt thereof, advantageously undergoes hydrolysis in vivo thereby providing the corresponding active drug compound of formula (I). Alkaline phosphatase is an enzyme that is associated with the conversion of the compound of the present disclosure, or a pharmaceutically acceptable salt thereof, to its active form upon hydrolysis.

In particular, it has been surprisingly found that the presence of the phosphoric acid ester moicty in the compound of the present disclosure allows salts to form which advantageously induce crystalline behavior in the pro-drug. The resulting crystalline form of the compound of the present disclosure enables reliable and consistent manufacturing processes, as well as improved stability and storage characteristics.

Furthermore, the phosphoric acid ester moiety advantageously imparts water solubility to the compound of the present disclosure, enabling its formulation in aqueous dosage forms. This enhanced solubility facilitates better drug absorption, distribution, and bioavailability, leading to improved therapeutic outcomes. Therefore, the compound of the present disclosure, or pharmaceutically acceptable salts thereof, are particularly suitable for the preparation of medicaments due to their improved aqueous solubility and crystallinity, ultimately benefiting the development of effective and stable pharmaceutical formulations.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Compounds

In one aspect, disclosed herein is a compound which is a phosphoric acid ester derivative of a compound of formula (I):

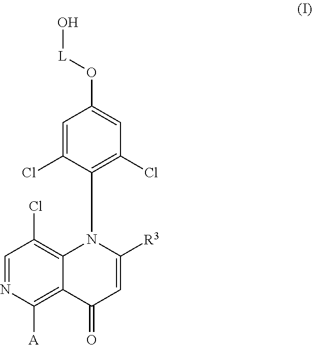

or a pharmaceutically acceptable salt thereof, wherein
L is (C₁-C₆) alkylene optionally substituted with one or more —OH;
A is —OR² or (C₁-C₆)alkyl optionally substituted with one or more substituents independently selected from —SO₂(C₁-C₄)alkyl, —NHC(O)R^{a}, and —C(O)NHR^{b};
R² is (C₁-C₆) alkyl substituted with one or more substituents independently selected from —NHC(O)R^{c} and —C(O)NHR^{d}, wherein the (C₁-C₆) alkyl is further optionally substituted with one or more substituents independently selected from halogen, —OH and —CN;
R^{a}, R^{b}, R^{c}, and R^{d} are each independently selected from H and (C₁-C₆) alkyl optionally substituted with one or more —OH; and
R³ is (C₁-C₄) alkyl.

In one embodiment, disclosed herein is a compound of formula (II):

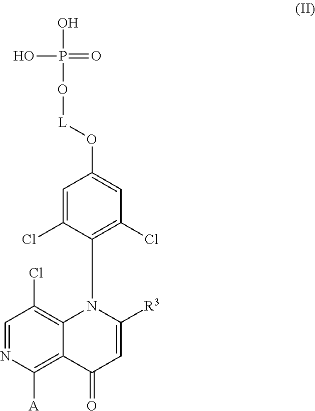

or a pharmaceutically acceptable salt thereof, wherein
L is (C₁-C₆)alkylene optionally substituted with one or more —OH;
A is —OR² or (C₁-C₆)alkyl optionally substituted with one or more substituents independently selected from —SO₂(C₁-C₄)alkyl, —NHC(O)R^{a}, and —C(O)NHR^{b};
R² is (C₁-C₆)alkyl substituted with one or more substituents independently selected from —NHC(O)R^{c} and —C(O)NHR^{d}, wherein the (C₁-C₆)alkyl is optionally further substituted with one or more substituents independently selected from halogen, —OH and —CN;
R^{a}, R^{b}, R^{c} and R^{d} are each independently selected from H and (C₁-C₆)alkyl optionally substituted with one or more —OH; and
R³ is (C₁-C₄)alkyl.

In another embodiment, the present disclosure provides a compound of formula (II-A):

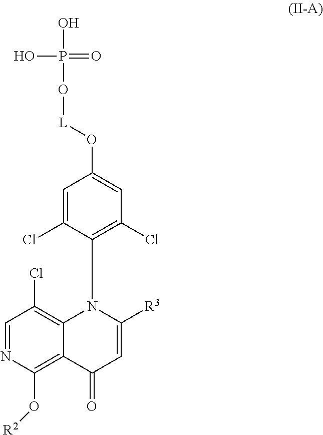

or a pharmaceutically acceptable salt thereof, wherein
L is (C₁-C₆)alkylene optionally substituted with one or more —OH;
R² is (C₁-C₆)alkyl substituted with one or more substituents independently selected from —NHC(O)R^{c} and —C(O)NHR^{d}, wherein the (C₁-C₆)alkyl is optionally further substituted with one or more substituents independently selected from halogen, —OH and —CN;
R^{c} and R^{d} are each independently selected from H and (C₁-C₆)alkyl optionally substituted with one or more —OH; and
R³ is (C₁-C₄)alkyl.

In another embodiment, the present disclosure provides a compound of formula (II-A), or a pharmaceutically acceptable salt thereof, as defined herein wherein
L is (C₁-C₆)alkylene optionally substituted with one or more —OH;
R² is (C₁-C₆) alkyl substituted with one or more substituents independently selected from —NHC(O)R^{c} and —C(O)NHR^{d}, wherein the (C₁-C₆)alkyl is further substituted with one or more —OH;
R^{c} and R^{d} are each independently selected from H and (C₁-C₆) alkyl optionally substituted with one or more —OH; and
R³ is (C₁-C₄)alkyl.

In one particular aspect of any one of the embodiments of the present disclosure, the present disclosure provides a compound of formula (III):

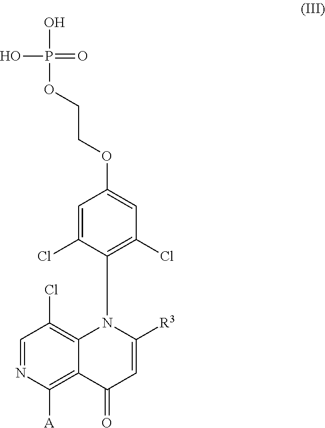

or a pharmaceutically acceptable salt thereof, wherein
A is —OR² or (C₁-C₆)alkyl optionally substituted with one or more substituents independently selected from —SO₂(C₁-C₄)alkyl, —NHC(O)R^{a}, and —C(O)NHR^{b};
R² is (C₁-C₆) alkyl substituted with one or more substituents independently selected from —NHC(O)R^{c} and —C(O)NHR^{d}, wherein the (C₁-C₆)alkyl is optionally further substituted with one or more substituents independently selected from halogen, —OH and —CN;
R^{a}, R^{b}, R^{c}, and R^{d} are each independently selected from H and (C₁-C₆)alkyl optionally substituted with one or more —OH; and
R³ is (C₁-C₄)alkyl.

In one embodiment, the present disclosure provides a compound of formula (III-A):

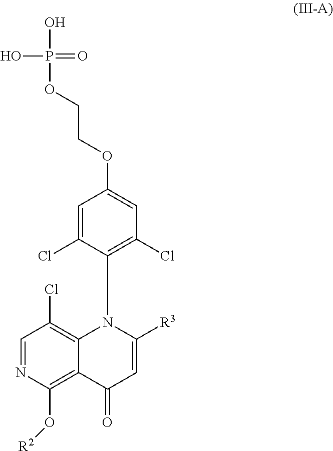

or a pharmaceutically acceptable salt thereof, wherein
R² is (C₁-C₆) alkyl substituted with one or more substituents independently selected from —NHC(O)R^{c} and —C(O)NHR^{d}, wherein the (C₁-C₆)alkyl is optionally further substituted with one or more substituents independently selected from halogen, —OH and —CN;
R^{c} and R^{d} are each independently selected from H and (C₁-C₆)alkyl optionally substituted with one or more —OH; and
R³ is (C₁-C₄)alkyl.

In another embodiment, the present disclosure provides a compound of formula (III-A), or a pharmaceutically acceptable salt thereof, as defined herein wherein
R² is (C₁-C₆)alkyl substituted with one or more substituents independently selected from —NHC(O)R^{c} and —C(O)NHR^{d}, wherein the (C₁-C₆) alkyl is further substituted with one or more substituents independently selected from halogen, —OH and —CN;
R^{c} and R^{d} are each independently selected from H and (C₁-C₆) alkyl optionally substituted with one or more —OH; and
R³ is (C₁-C₄)alkyl.

In another embodiment, the present disclosure provides a compound of formula (III-B):

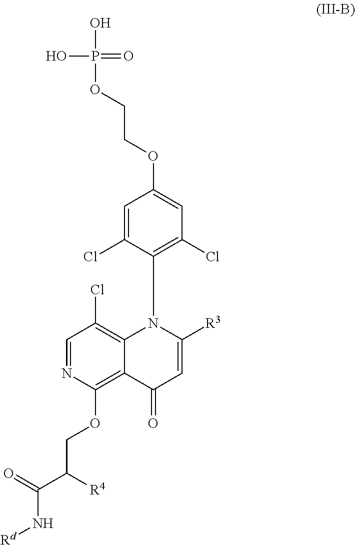

or a pharmaceutically acceptable salt thereof, wherein
R^{d} is selected from H and (C₁-₆)alkyl optionally substituted with one or more —OH;
R³ is (C₁-C₄)alkyl; and
R⁴ is selected from H, halogen, —OH and —CN.

In another embodiment, the present disclosure provides a compound of formula (III-C):

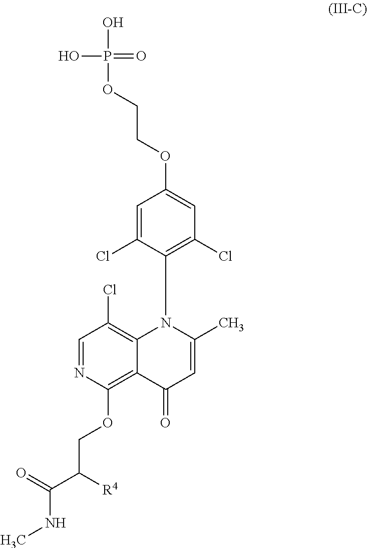

or a pharmaceutically acceptable salt thereof, wherein
R⁴ is H or —OH.

In another particular aspect of any one of the embodiments of the present disclosure, the present disclosure provides a compound of formula (IV):

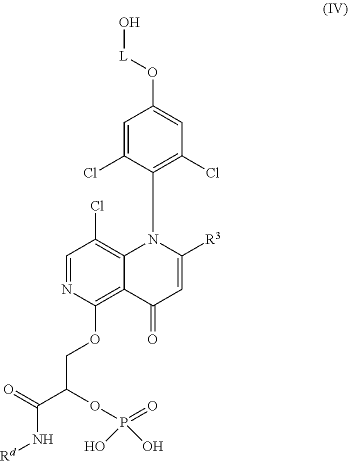

or a pharmaceutically acceptable salt thereof, wherein
L is (C₁-C₆)alkylene optionally substituted with one or more —OH;
R^{d} is selected from H and (C₁-C₆)alkyl optionally substituted with one or more —OH; and
R³ is (C₁-C₄)alkyl.

In one embodiment, the present disclosure provides a compound of formula (IV-A):

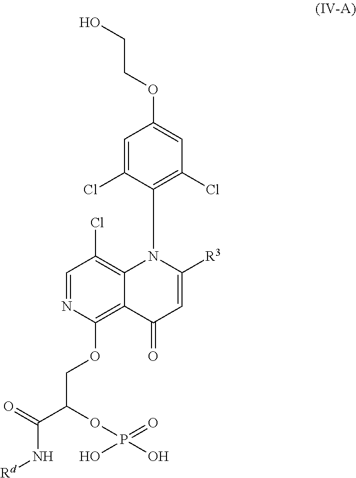

or a pharmaceutically acceptable salt thereof, wherein
R^{d} is selected from H and (C₁-C₆)alkyl optionally substituted with one or more —OH; and
R³ is (C₁-C₄)alkyl.

In another embodiment, the present disclosure provides a compound of formula (IV-A), or a pharmaceutically acceptable salt thereof, as defined herein wherein
R^{d} is (C₁-C₆)alkyl optionally substituted with one or more —OH, preferably methyl; and
R³ is (C₁-C₄)alkyl, preferably methyl.

In another particular aspect of any one of the embodiments of the present disclosure, the present disclosure provides a compound of formula (V):

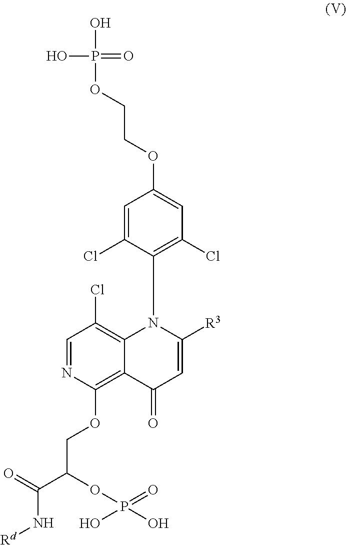

or a pharmaceutically acceptable salt thereof, wherein
R^{d} is selected from H and (C₁-C₆) alkyl optionally substituted with one or more —OH; and
R³ is (C₁-C₄)alkyl.

In one embodiment, the present disclosure provides a compound of formula (V), or a pharmaceutically acceptable salt thereof, as defined herein wherein
R^{d} is (C₁-C₆) alkyl optionally substituted with one or more —OH, preferably methyl; and
R³ is (C₁-C₄)alkyl, preferably methyl.

The compound of the present disclosure including the compound of any of formulae (II), (II-A), (III), (III-A), (III-B), (III-C), (IV), (IV-A) or (V), or a pharmaceutically acceptable salt thereof, as defined herein is suitable for use as pro-drug of the compound of formula (I), or a pharmaceutically acceptable salt thereof, as defined hercin.

As used herein, the term “pro-drug” refers to a compound which converts in vivo to an inhibitor of GIRK1/4. A pro-drug is an active or inactive compound that is modified chemically through in vivo physiological action, such as hydrolysis, metabolism and the like, into a compound of this disclosure following administration of the pro-drug to a subject. The suitability and techniques involved in making and using pro-drugs are well known by those skilled in the art. Pro-drugs can be conceptually divided into two non-exclusive categories, bioprecursor pro-drugs and carrier pro-drugs. See The Practice of Medicinal Chemistry, Ch. 31-32 (Ed. Wermuth, Academic Press, San Diego, Calif., 2001).

Generally, bioprecursor pro-drugs are compounds which are inactive or have low activity compared to the corresponding active drug compound, that contain one or more protective groups and are converted to an active form by metabolism or solvolysis. Both the active drug form and any released metabolic products should have acceptably low toxicity.

Carrier pro-drugs are drug compounds that contain a transport moiety, e.g., that improve uptake and/or localized delivery to a site(s) of action. Desirably for such a carrier pro-drug, the linkage between the drug moiety and the transport moiety is a covalent bond, the pro-drug is inactive or less active than the drug compound. and any released transport moiety is acceptably non-toxic. For pro-drugs where the transport moiety is intended to enhance uptake, typically the release of the transport moiety should be rapid. In other cases. it is desirable to utilize a moiety that provides slow release, e.g., certain polymers or other moieties, such as cyclodextrins. Carrier pro-drugs can, for example, be used to improve one or more of the following properties: increased lipophilicity, increased duration of pharmacological effects. increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Unless specified otherwise, the term “compounds of the present disclosure” or “compound of the present disclosure” refers to phosphate ester derivatives of the compounds of formula (I) as well as compounds of any of formulae (II), (II-A), (III), (III-A), (III-B), (III-C), (IV), (IV-A) or (V), and exemplified compounds, or pharmaceutically acceptable salts thereof, as well as all stereoisomers (including diastereoisomers and enantiomers), rotamers, tautomers, hydrates, solvates, polymorphs, co-crystals, and isotopically labeled compounds (including deuterium substitutions), as well as inherently formed moieties.

Various embodiments of the disclosure are described herein. It will be recognized that features specified in each embodiment may be combined with other specified features of other embodiments to provide further embodiments.

In some embodiments, L is (C₁-C₄)alkylene optionally substituted with one or more —OH. In certain embodiments, L is selected from —CH₂CH₂—, —CH₂—CH(CH₃)—, and —CH₂C(CH₃)₂—. In some embodiments, L is selected from

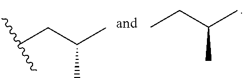

In some embodiments, L is —CH₂CH₂—.

In some embodiments, A is —OR².

In some embodiments, R² is (C₁-C₆)alkyl substituted with one or more substituents independently selected from —NHC(O)CH₃, —C(O)NHCH₃, —C(O)NHCH₂CH₂OH, and —C(O)NH₂, and wherein the (C₁-C₆)alkyl is further optionally substituted with one or more substituents independently selected from halogen, —OH and —CN. In some embodiments, R² is (C₁-C₄)alkyl substituted with one or more substituents independently selected from —NHC(O)CH₃, —C(O)NHCH₃, —C(O)NHCH₂CH₂OH, and —C(O)NH₂, and wherein the (C₁-C₄)alkyl is further optionally substituted with one or more substituents independently selected from halogen, —OH and —CN. In some embodiments, R² is (C₁-C₄)alkyl substituted with one or more substituents independently selected from —NHC(O)CH₃, —C(O)NHCH₃, —C(O)NHCH₂CH—OH, and —C(O)NH₂, and wherein the (C₁-C₄)alkyl is further optionally substituted with one or more substituents independently selected from halogen and —OH. In some embodiments, R² is (C₁-C₄)alkyl substituted with one or more substituents independently selected from —NHC(O)CH₃, —C(O)NHCH₃, —C(O)NHCH₂CH₂OH, and —C(O)NH₂, wherein the (C₁-C₄)alkyl is further optionally substituted with one or more substituents independently selected from fluoro and —OH.

In certain embodiments, R² is selected from

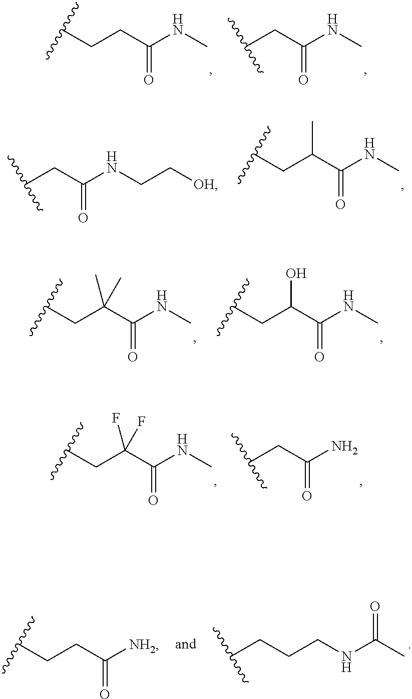

In some embodiments, R² is selected from

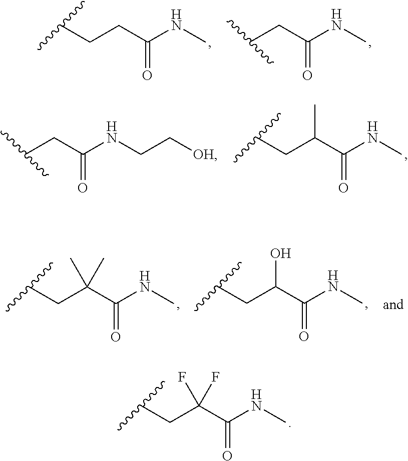

In some embodiments, R² is selected from

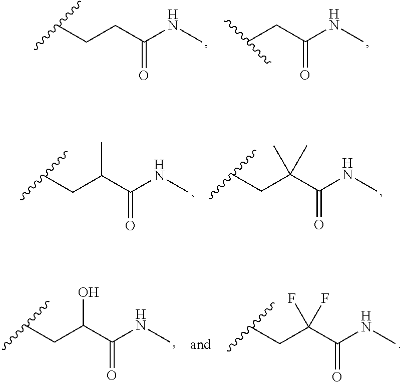

In some embodiments, R² is selected from

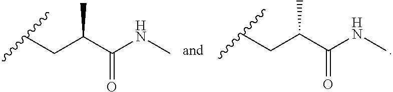

In some embodiments, R² is selected from

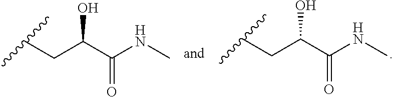

In some embodiments, A is (C₁-C₆)alkyl optionally substituted with one or more substituents independently selected from —SO₂CH₃ and —NHC(O)CH₃. In some embodiments, A is (C₁-C₄)alkyl optionally substituted with one or more substituents independently selected from —SO₂CH₃ and —NHC(O)CH₃.

In certain embodiments, A is selected from

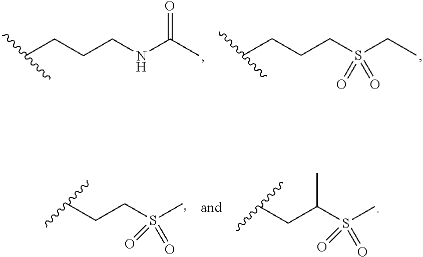

In certain embodiments, A is selected from

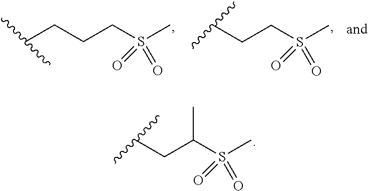

In certain embodiments, A is

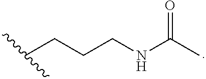 [SMILES: *CCCNC(C)=O]

In some embodiments, R³ is selected from H and (C₁-C₆)alkyl. In some embodiments, R³ is selected from H and (C₁-C₄)alkyl. In some embodiments, R³ is selected from —CH₃ and —CH₂CH₃. In some embodiments, R³ is —CH₃.

In some embodiments, R^{a} is selected from H and (C₁-C₆)alkyl. In some embodiments, R^{b} is selected from H and (C₁-C₆)alkyl. In some embodiments, R^{c} is selected from H and (C₁-C₆)alkyl. In some embodiments, R^{a} is selected from H and (C₁-C₄)alkyl. In some embodiments, R^{b} is selected from H and (C₁-C₄)alkyl. In some embodiments, R^{c} is selected from H and (C₁-C₄)alkyl. In some embodiments, R^{d} is selected from H and (C₁-C₄)alkyl optionally substituted with one or more —OH. In some embodiments, R^{d} is selected from —CH₃ and —CH₂CH₃. In some embodiments, R^{d} is —CH₃.

In certain embodiments, the compound of formula (I) of the present disclosure is a compound or a pharmaceutically acceptable salt thereof selected from the group consisting of:

| Compound | Structure | Chemical Name |
|---|---|---|
| C-1 | 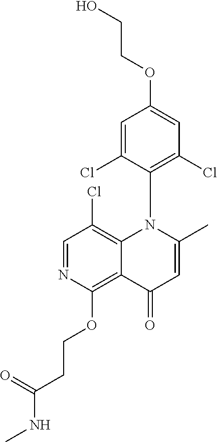 [SMILES: CNC(=O)CCOc1ncc(Cl)c2c1c(=O)cc(C)n2-c1c(Cl)cc(OCCO)cc1Cl] | 3-((8-chloro-1-(2,6-dichloro-4-(2- hydroxyethoxy)phenyl)-2-methyl- 4-oxo-1,4-dihydro-1,6- naphthyridin-5-yl)oxy)-N- methylpropanamide |
| C-2 | 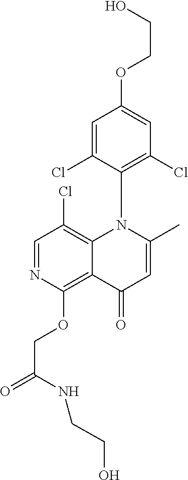 [SMILES: Cc1cc(=O)c2c(OCC(=O)NCCO)ncc(Cl)c2n1-c1c(Cl)cc(OCCO)cc1Cl] | 2-((8-chloro-1-(2,6-dichloro-4-(2- hydroxyethoxy)phenyl)-2-methyl- 4-oxo-1,4-dihydro-1,6- naphthyridin-5-yl)oxy)-N-(2- hydroxyethyl)acetamide |
| C-3 | 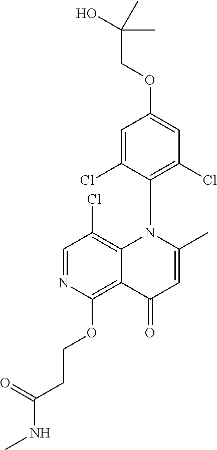 [SMILES: CNC(=O)CCOc1ncc(Cl)c2c1c(=O)cc(C)n2-c1c(Cl)cc(OCC(C)(C)O)cc1Cl] | 3-((8-chloro-1-(2,6-dichloro-4-(2- hydroxy-2- methylpropoxy)phenyl)-2-methyl- 4-oxo-1,4-dihydro-1,6- naphthyridin-5-yl)oxy)-N- methylpropanamide |
| C-4 | 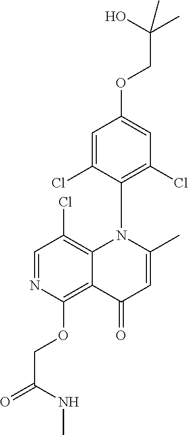 [SMILES: CNC(=O)COc1ncc(Cl)c2c1c(=O)cc(C)n2-c1c(Cl)cc(OCC(C)(C)O)cc1Cl] | 2-((8-chloro-1-(2,6-dichloro-4-(2- hydroxy-2- methylpropoxy)phenyl)-2-methyl- 4-oxo-1,4-dihydro-1,6- naphthyridin-5-yl)oxy)-N- methylacetamide |
| C-5 | 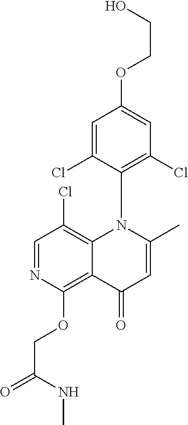 [SMILES: CNC(=O)COc1ncc(Cl)c2c1c(=O)cc(C)n2-c1c(Cl)cc(OCCO)cc1Cl] | 2-((8-chloro-1-(2,6-dichloro-4-(2- hydroxyethoxy)phenyl)-2-methyl- 4-oxo-1,4-dihydro-1,6- naphthyridin-5-yl)oxy)-N- methylacetamide |
| C-6 | 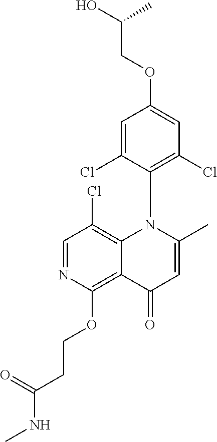 [SMILES: CNC(=O)CCOc1ncc(Cl)c2c1c(=O)cc(C)n2-c1c(Cl)cc(OC[C@@H](C)O)cc1Cl] | (R)-3-((8-chloro-1-(2,6-dichloro- 4-(2-hydroxypropoxy)phenyl)-2- methyl-4-oxo-1,4-dihydro-1,6- naphthyridin-5-yl)oxy)-N- methylpropanamide |
| C-7 | 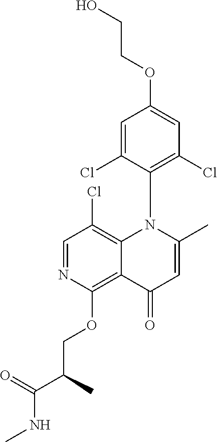 [SMILES: CNC(=O)[C@H](C)COc1ncc(Cl)c2c1c(=O)cc(C)n2-c1c(Cl)cc(OCCO)cc1Cl] | (R)-3-((8-chloro-1-(2,6-dichloro- 4-(2-hydroxyethoxy)phenyl)-2~ methyl-4-oxo-1,4-dihydro-1,6- naphthyridin-5-yl)oxy)-N,2- dimethylpropanamide |
| C-8 | 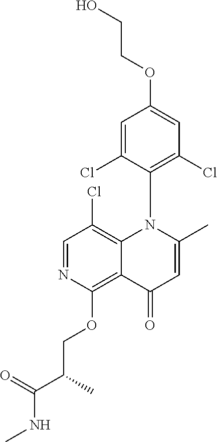 [SMILES: CNC(=O)[C@@H](C)COc1ncc(Cl)c2c1c(=O)cc(C)n2-c1c(Cl)cc(OCCO)cc1Cl] | (S)-3-((8-chloro-1-(2,6-dichloro- 4-(2-hydroxyethoxy)phenyl)-2- methyl-4-oxo-1,4-dihydro-1,6- naphthyridin-5-yl)oxy)-N,2- dimethylpropanamide |
| C-9 | 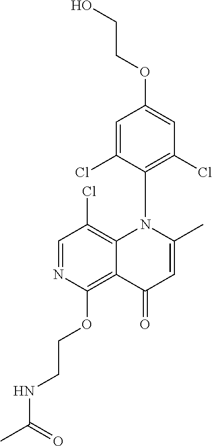 [SMILES: CC(=O)NCCOc1ncc(Cl)c2c1c(=O)cc(C)n2-c1c(Cl)cc(OCCO)cc1Cl] | N-(2-((8-chloro-1-(2,6-dichloro-4- (2-hydroxyethoxy)phenyl)-2- methyl-4-oxo-1,4-dihydro-1,6- naphthyridin-5- yl)oxy)ethyl)acetamide |
| C-10 | 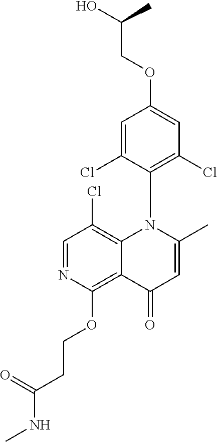 [SMILES: CNC(=O)CCOc1ncc(Cl)c2c1c(=O)cc(C)n2-c1c(Cl)cc(OC[C@H](C)O)cc1Cl] | (S)-3-((8-chloro-1-(2,6-dichloro- 4-(2-hydroxypropoxy)phenyl)-2- methyl-4-oxo-1,4-dihydro-1,6- naphthyridin-5-yl)oxy)-N- methylpropanamide |
| C-11 | 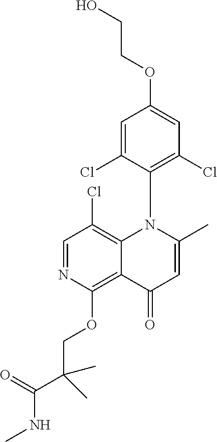 [SMILES: CNC(=O)C(C)(C)COc1ncc(Cl)c2c1c(=O)cc(C)n2-c1c(Cl)cc(OCCO)cc1Cl] | 3-((8-chloro-1-(2,6-dichloro-4-(2- hydroxyethoxy)phenyl)-2-methyl- 4-oxo-1,4-dihydro-1,6- naphthyridin-5-yl)oxy)-N,2,2- trimethylpropanamide |
| C-12 | 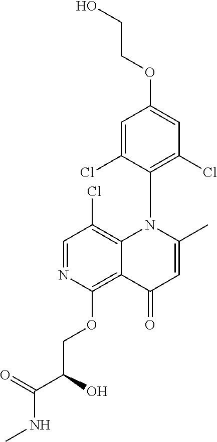 [SMILES: CNC(=O)[C@H](O)COc1ncc(Cl)c2c1c(=O)cc(C)n2-c1c(Cl)cc(OCCO)cc1Cl] | (R)-3-((8-chloro-1-(2,6-dichloro- 4-(2-hydroxyethoxy)phenyl)-2~ methyl-4-oxo-1,4-dihydro-1,6- naphthyridin-5-yl)oxy)-2- hydroxy-N-methylpropanamide |
| C-13 | 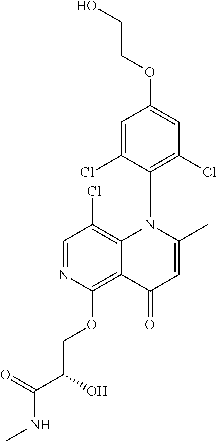 [SMILES: CNC(=O)[C@@H](O)COc1ncc(Cl)c2c1c(=O)cc(C)n2-c1c(Cl)cc(OCCO)cc1Cl] | (S)-3-((8-chloro-1-(2,6-dichloro- 4-(2-hydroxyethoxy)phenyl)-2- methyl-4-oxo-1,4-dihydro-1,6- naphthyridin-5-yl)oxy)-2- hydroxy-N-methylpropanamide |
| C-14 | 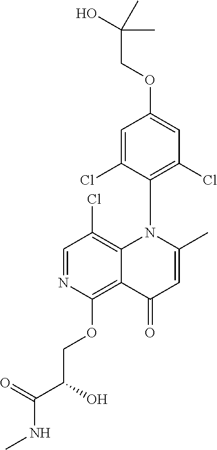 [SMILES: CNC(=O)[C@@H](O)COc1ncc(Cl)c2c1c(=O)cc(C)n2-c1c(Cl)cc(OCC(C)(C)O)cc1Cl] | (S)-3-((8-chloro-1-(2,6-dichloro- 4-(2-hydroxy-2~ methylpropoxy)phenyl)-2-methyl- 4-oxo-1,4-dihydro-1,6- naphthyridin-5-yl)oxy)-2- hydroxy-N-methylpropanamide |
| C-15 | 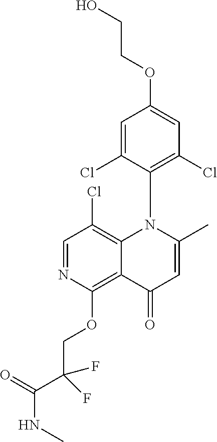 [SMILES: CNC(=O)C(F)(F)COc1ncc(Cl)c2c1c(=O)cc(C)n2-c1c(Cl)cc(OCCO)cc1Cl] | 3-((8-chloro-1-(2,6-dichloro-4-(2- hydroxyethoxy)phenyl)-2-methyl- 4-oxo-1,4-dihydro-1,6- naphthyridin-5-yl)oxy)-2,2- difluoro-N-methylpropanamide |
| C-16 | 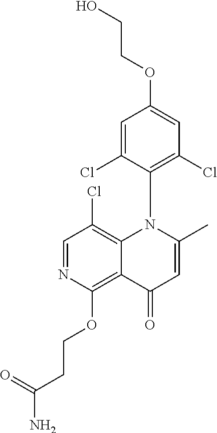 [SMILES: Cc1cc(=O)c2c(OCCC(N)=O)ncc(Cl)c2n1-c1c(Cl)cc(OCCO)cc1Cl] | 3-((8-chloro-1-(2,6-dichloro-4-(2- hydroxyethoxy)phenyl)-2-methyl- 4-oxo-1,4-dihydro-1,6- naphthyridin-5- yl)oxy)propanamide |
| C-17 | 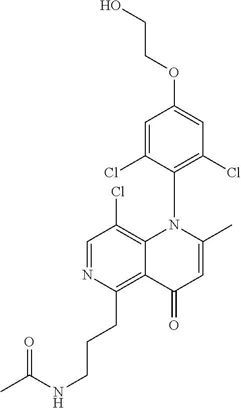 [SMILES: CC(=O)NCCCc1ncc(Cl)c2c1c(=O)cc(C)n2-c1c(Cl)cc(OCCO)cc1Cl] | N-(3-(8-chloro-1-(2,6-dichloro-4- (2-hydroxyethoxy)phenyl)-2- methyl-4-oxo-1,4-dihydro-1,6- naphthyridin-5- yl)propyl)acetamide |
| C-18 | 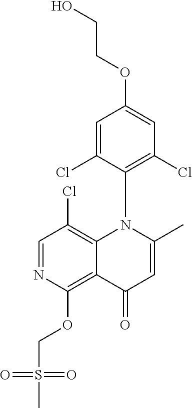 [SMILES: Cc1cc(=O)c2c(OCS(C)(=O)=O)ncc(Cl)c2n1-c1c(Cl)cc(OCCO)cc1Cl] | 8-chloro-1-(2,6-dichloro-4-(2- hydroxyethoxy)phenyl)-2-methyl- 5-(2-(methylsulfonyl)ethyl)-1,6- naphthyridin-4(1H)-one |
| C-19 | 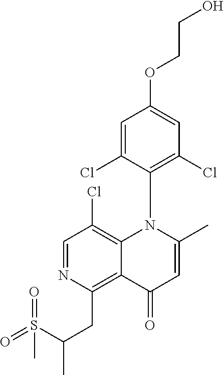 [SMILES: Cc1cc(=O)c2c(CC(C)S(C)(=O)=O)ncc(Cl)c2n1-c1c(Cl)cc(OCCO)cc1Cl] | 8-chloro-1-(2,6-dichloro-4-(2- hydroxyethoxy)phenyl)-2-methyl- 5-(2-(methylsulfonyl)propyl)-1,6- naphthyridin-4(1H)-one |
| C-20 | 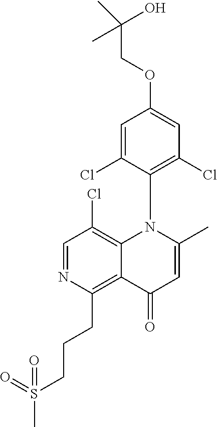 [SMILES: Cc1cc(=O)c2c(CCCS(C)(=O)=O)ncc(Cl)c2n1-c1c(Cl)cc(OCC(C)(C)O)cc1Cl] | 8-chloro-1-(2,6-dichloro-4-(2- hydroxy-2- methylpropoxy)phenyl)-2-methyl- 5-(3-(methylsulfonyl)propyl)-1,6- naphthyridin-4(1H)-one |

In certain embodiments, the compound of the present disclosure, or a pharmaceutically acceptable salt thereof, as defined herein is a pro-drug of any one of the compounds C-1 to C-20, or a pharmaceutically acceptable salt thereof, as defined herein.

In certain embodiments, the compound of the present disclosure, or a pharmaceutically acceptable salt thereof, as defined herein is selected from the group consisting of:

| | | |
|---|---|---|
| P-1 | 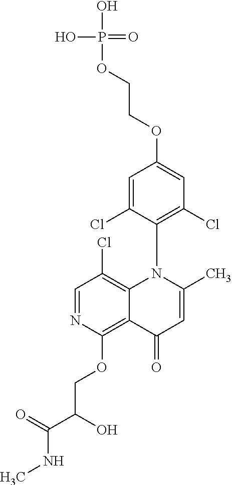 [SMILES: CNC(=O)C(O)COc1ncc(Cl)c2c1c(=O)cc(C)n2-c1c(Cl)cc(OCCOP(=O)(O)O)cc1Cl] | 2-(3,5-dichloro-4- (8-chloro-5-(2- hydroxy-3- (methylamino)-3- oxopropoxy)-2- methyl-4-oxo-1,6- naphthyridin-1(4H)- yl)phenoxy)ethyl dihydrogen phosphate |
| P-2 | 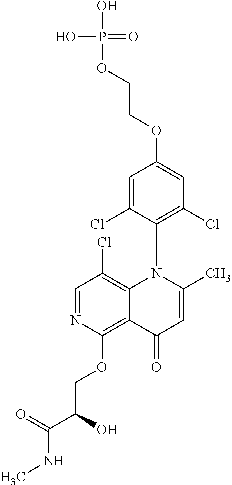 [SMILES: CNC(=O)[C@H](O)COc1ncc(Cl)c2c1c(=O)cc(C)n2-c1c(Cl)cc(OCCOP(=O)(O)O)cc1Cl] | (R)-2-(3,5-dichloro- 4-(8-chloro-5- (2-hydroxy-3- (mcthylamino)-3- oxopropoxy)-2- methyl-4-oxo-1,6- naphthyridin-1(4H)- yl)phenoxy)ethyl dihydrogen phosphate |
| P-3 | 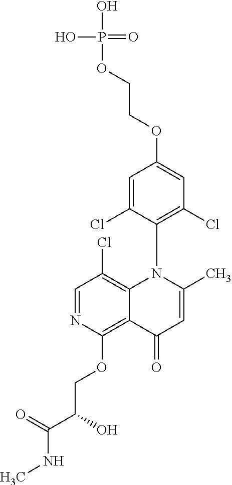 [SMILES: CNC(=O)[C@@H](O)COc1ncc(Cl)c2c1c(=O)cc(C)n2-c1c(Cl)cc(OCCOP(=O)(O)O)cc1Cl] | (S)-2-(3,5-dichloro- 4-(8-chloro-5- (2-hydroxy-3- (mcthylamino)-3~ oxopropoxy)-2- methyl-4-oxo-1,6- naphthyridin-1(4H)- yl)phenoxy)ethyl dihydrogen phosphate |
| P-4 | 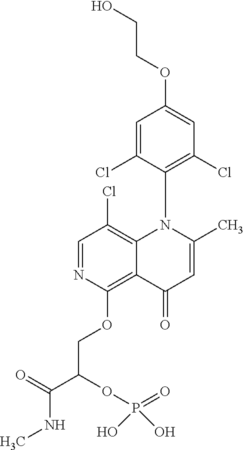 [SMILES: CNC(=O)C(COc1ncc(Cl)c2c1c(=O)cc(C)n2-c1c(Cl)cc(OCCO)cc1Cl)OP(=O)(O)O] | 3-((8-chloro-1- (2,6-dichloro-4-(2- hydroxyethoxy) phenyl)-2-methyl- 4-oxo-1,4- dihydro-1,6- naphthyridin-5- yl)oxy)-1- (methylamino)-1- oxopropan-2-yl dihydrogen phosphate |
| P-5 | 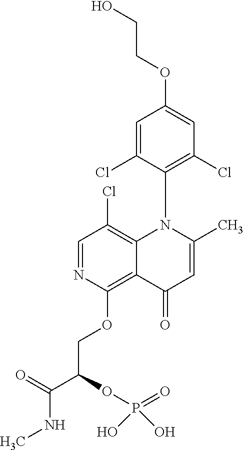 [SMILES: CNC(=O)[C@@H](COc1ncc(Cl)c2c1c(=O)cc(C)n2-c1c(Cl)cc(OCCO)cc1Cl)OP(=O)(O)O] | (R)-3-((8-chloro- 1-(2,6-dichloro- 4-(2-hydroxyethoxy) phenyl)-2- methyl-4-oxo- 1,4-dihydro-1,6- naphthyridin-5- yl)oxy)-1- (methylamino)- 1-oxopropan-2-yl dihydrogen phosphate |
| P-6 | 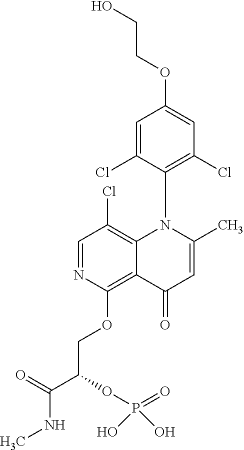 [SMILES: CNC(=O)[C@H](COc1ncc(Cl)c2c1c(=O)cc(C)n2-c1c(Cl)cc(OCCO)cc1Cl)OP(=O)(O)O] | (S)-3-((8-chloro- 1-(2,6-dichloro- 4-(2-hydroxyethoxy) phenyl)-2- methyl-4-oxo-1,4- dihydro-1,6- naphthyridin-5-yl) oxy)-1- (methylamino)-1- oxopropan-2-yl dihydrogen phosphate |

As used herein, the terms “salt” or “salts” refers to an acid addition or base addition salt of a compound of the present disclosure. “Salts” include in particular “pharmaceutical acceptable salts”. The term “pharmaceutically acceptable salts” refers to salts that retain the biological effectiveness and properties of the compounds of this disclosure and, which typically are not biologically or otherwise undesirable. In many cases, the compounds of the present disclosure are capable of forming acid and/or base salts by virtue of the presence of amino and/or carboxyl groups or groups similar thereto. When both a basic group and an acid group are present in the same molecule, the compounds of the present disclosure may also form internal salts, e.g., zwitterionic molecules.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include. for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present disclosure provides compounds of the present disclosure in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the disclosure include, for example, isotopes of hydrogen.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present disclosure. The concentration of deuterium may be defined by the isotopic enrichment factor. The term “isotopic enrichment factor” as used herein means the ratio between the isotopic abundance and the natural abundance of a specified isotope. If a substituent in a compound of this disclosure is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the disclosure include isotopes of hydrogen, carbon, nitrogen, oxygen, phosphorous, fluorine, and chlorine, such as ³H, ¹¹C, ¹³C, ¹⁴C, ¹⁵N, ¹⁸F ³¹P, ³²P, ³⁵S, ³⁶Cl, ¹²³I, ¹²⁴I, ¹²⁵I respectively. Accordingly, it should be understood that the disclosure includes compounds that incorporate one or more of any of the aforementioned isotopes, including for example, radioactive isotopes, such as ³H and ¹⁴C, or those into which non-radioactive isotopes, such as ³H and ¹³C are present. Such isotopically labelled compounds are useful in metabolic studies (with ¹⁴C), reaction kinetic studies (with, for example, ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present disclosure can generally be prepared by conventional techniques known to those skilled in the art or by processes analogous to those described in the accompanying Examples and General Synthetic Schemes using an appropriate isotopically-labeled reagents in place of the non-labeled reagent previously employed.

Any asymmetric atom (e.g., carbon or the like) of the compound(s) of the present disclosure can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present disclosure can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastercomers, racemates, for example, by chromatography and/or fractional crystallization.

Any resulting racemates of compounds of the present disclosure or of intermediates can be resolved into the optical antipodes by known methods, e.g., by separation of the diastereomeric salts thereof, obtained with an optically active acid or base, and liberating the optically active acidic or basic compound. In particular, a basic moicty may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the present disclosure or racemic intermediates can also be resolved by chiral chromatography, e.g., high pressure liquid chromatography (HPLC) using a chiral adsorbent.

The compounds of the present disclosure as defined herein, including free forms, pharmaceutically acceptable salts, hydrates and solvates thereof, may under the appropriate conditions be isolated in one or more crystalline forms.

Hence, in another aspect, the present disclosure related to a crystalline form of a phosphoric acid ester derivative of the compound of formula (I), or a pharmaceutically acceptable salt thereof, as defined herein.

The term “crystalline form” as used herein include reference to anhydrous crystalline forms. bydrate crystalline forms, solvate crystalline forms and mixtures of crystalline forms.

In one embodiment, a crystalline form of the compound of the present disclosure, or a pharmaceutically acceptable salt thereof, as defined herein is selected from a free form, a hydrate, a solvate, a polymorph and a co-crystal thereof.

The term “free form” as used herein refers to the compound of the present disclosure as defined herein per se without salt formation or association with a solvent (e.g., solvate).

The term “hydrate” as used herein refers to a crystalline form containing one or more water molecules in a three-dimensional periodic arrangement. It can include non-stoichiometric hydrates or stoichiometric hydrates, such as hemihydrates, monohydrates, dihydrates and trihydrates.

The term “solvate” as used herein refers to a crystalline form containing one or more solvent molecules other than water in a three-dimensional periodic arrangement. The solvate may comprise either a stoichiometric or non-stoichiometric amount of the solvent molecules.

In one embodiment, the compounds of the present disclosure, or pharmaceutically acceptable salts thereof, as defined herein may inherently or by design form hydrates or solvates with pharmaceutically acceptable solvents.

The term “polymorph” as used herein refers to crystalline forms having the same chemical composition but different spatial arrangements of the molecules, atoms, and/or ions forming the crystal.

In one embodiment, compounds of the present disclosure, or pharmaceutically acceptable salts thereof, as defined herein that contain groups capable of acting as donors and/or acceptors for hydrogen bonds may be capable of forming co-crystalline forms with suitable co-crystal formers.

The terms “co-crystalline” and “co-crystal” are used herein interchangeably to mean a co-crystal comprising a compound of the present disclosure, or a pharmaceutically acceptable salt thereof, as defined herein and a suitable co-crystal former.

These co-crystals may be prepared from the compounds of the present disclosure, or pharmaceutically acceptable salts thereof, as defined berein by known co-crystal forming procedures such as, e.g., grinding, heating, co-subliming, co-melting, or contacting in solution the compounds of the present disclosure. or pharmaceutically acceptable salts thereof, with a co-crystal former under crystallization conditions and isolating co-crystals thereby formed. Suitable co-crystal formers include, for instance, those described in WO 2004/078163.

In one embodiment, a crystalline form of the compound of the present disclosure, or a pharmaceutically acceptable salt thereof, as defined herein is provided in substantially pure form. As used herein, “substantially pure,” when used in reference to a crystalline form, means a compound having a purity greater than 90% by weight, including greater than 90, 91, 92, 93, 94, 95, 96, 97, 98, and 99% by weight. and also including equal to about 100% by weight of a compound of the present disclosure or a pharmaceutically acceptable salt thereof, based on the weight of the compound. The remaining material comprises other form(s) of the compound of the present disclosure, and/or reaction impurities and/or processing impurities arising from its preparation. For example, a crystalline form of a compound of the present disclosure. or a pharmaceutically acceptable salt thereof, as defined herein may be deemed substantially pure in that it has a purity greater than 90% by weight, as measured by means that are at this time known and generally accepted in the art, where the remaining less than 10% by weight of material comprises other form(s) of the compound of the present disclosure and/or reaction impurities and/or processing impurities.

A particular crystalline form of the compound of the present disclosure, or a pharmaceutically acceptable salt thereof, as defined herein may be referred to as “crystalline form X”, “crystal form X”, “co-crystal form”, “polymorph form X”, “modification X”, or “H_{X″} where ‘X’ is the letter which is assigned to that particular crystalline form. The names used herein to characterize a specific crystalline form, e.g. “A-1” etc., should not be considered limiting with respect to any other substance possessing similar or identical physical and chemical characteristics, but rather it should be understood that these designations are mere identifiers that should be interpreted according to the characterization information also presented herein.

### Pharmaceutical Compositions

In another aspect, the present disclosure provides a pharmaceutical composition comprising a compound of the present disclosure, or a pharmaceutically acceptable salt thereof, and one or more pharmaceutically acceptable carriers. In a further embodiment, the composition comprises at least two pharmaceutically acceptable carriers, such as those described herein.

In some embodiments, a pharmaceutical composition further comprises at least one additional pharmaceutically active agent. In some embodiments, the additional pharmaceutically active agent is selected from Class I antiarrhythmic agents, Class II antiarrhythmic agents, Class III antiarrhythmic agents, Class IV antiarrhythmic agents, Class V antiarrhythmic agents, cardiac glycosides and other drugs affecting atrial refractoriness, haemostasis modulators, antithrombotics; thrombin inhibitors; factor VIIa inhibitors; anticoagulants, factor Xa inhibitors, and direct thrombin inhibitors; antiplatelet agents, cyclooxygenase inhibitors, adenosine diphosphate (ADP) receptor inhibitors, phosphodiesterase inhibitors, glycoprotein IIB/IIA, adenosine reuptake inhibitors; anti-dyslipidemia agents, HMG-COA reductase inhibitors, other cholesterol-lowering agents; bile acid sequestrants; cholesterol absorption inhibitors; cholesteryl ester transfer protein (CETP) inhibitors; inhibitors of the ileal bile acid transport system (IBAT inhibitors); bile acid binding resins; nicotinic acid and analogues thereof; anti-oxidants; omega-3 fatty acids; antihypertensive agents, including adrenergic receptor antagonists, beta blockers, alpha blockers, mixed alpha/beta blockers; adrenergic receptor agonists, alpha-2 agonists; angiotensin converting enzyme (ACE) inhibitors, calcium channel blockers; angiotensin II receptor antagonists, aldosterone receptor antagonists; centrally acting adrenergic drugs, central alpha agonists; and diuretic agents; anti-obesity agents, pancreatic lipase inhibitors, microsomal transfer protein (MTP) modulators, diacyl glycerolacyltransferase (DGAT) inhibitors, cannabinoid (CBI) receptor antagonists; insulin and insulin analogues; insulin secretagogues; agents that improve incretin action, dipeptidyl peptidase IV (DPP-4) inhibitors, glucagon-like peptide-1 (GLP-1) agonists; insulin sensitizing agents, peroxisome proliferator activated receptor gamma (PPARγ) agonists, agents that modulate hepatic glucose balance, fructose 1,6-bisphosphatase inhibitors, glycogen phosphorylase inhibitors, glycogen synthase kinase inhibitors, glucokinase activators; agents designed to reduce/slow the absorption of glucose from the intestine, alpha-glucosidase inhibitors; agents which antagonize the actions of or reduce secretion of glucagon, amylin analogues; agents that prevent the reabsorption of glucose by the kidney, and sodium-dependent glucose transporter 2 (SGLT-2) inhibitors and combinations thereof.

The pharmaceutical composition can be formulated for particular routes of administration such as oral administration, parenteral administration (e.g., by injection, infusion, transdermal or topical administration), and rectal administration. Topical administration may also pertain to inhalation or intranasal application. The pharmaceutical compositions of the present disclosure can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions, using polyalkylene glycols such as propylene glycol, as the carrier.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient, the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

The pharmaceutical composition or combination of the present disclosure may, for example, be in unit dosage of about 1-1000 mg of active ingredient(s) for a subject of about 50-70 kg. In one embodiment, the compositions are in the form of a tablet that can be scored. The therapeutically effective dosage of a compound, the pharmaceutical composition, or the combinations thereof, is dependent on the species of the subject, the body weight, age and individual condition, the disorder or disease or the severity thereof being treated.

### Methods of Use

In yet another aspect, the present disclosure is directed to a method for treating or preventing a disease or disorder comprising administering to a patient in need thereof a therapeutically effective amount of a compound of the present disclosure, or a pharmaceutically acceptable salt thereof, or a pharmaceutical composition comprising the compound of the present disclosure, or a pharmaceutically acceptable salt thereof, and a pharmaceutically acceptable carrier.

A method for treating or preventing a disease or disorder according to the present disclosure includes administering a therapeutically effective amount of a compound of the present disclosure, or a pharmaceutically acceptable salt thereof, or a pharmaceutical composition comprising the compound of the present disclosure, or a pharmaceutically acceptable salt thereof, and a pharmaceutically acceptable carrier to a patient in need thereof, said patient possessing an enzyme and a membrane transporter wherein the compound of the present disclosure is a substrate for both the enzyme and the membrane transporter.

In a particular embodiment, the method of the disclosure comprises the step of administering a therapeutically effective amount of a compound of the present disclosure, or a pharmaceutically acceptable salt thereof, or a pharmaceutical composition comprising the compound of the present disclosure, or a pharmaceutically acceptable salt thereof, and a pharmaceutically acceptable carrier to the gastrointestinal lumen of a patient in need thereof. The compound of the present disclosure is transported from the gastrointestinal lumen by a specific transporter and enzymatically cleaved to yield the corresponding compound of formula (I) thereby delivering the compound of formula (I) to the patient in need thereof.

Examples of enzymes which are particularly suitable for drug delivery include, inter alia, phosphatases, especially alkaline phosphatase.

In some embodiments, the disease or disorder is selected from cardiac arrhythmia, atrial fibrillation, bradyarrhythmia, bradycardia, heart block, sick sinus syndrome, parasympathetic hyperactivation, primary hyperaldosteronism, hypotension. and vasovagal syncope. In some embodiments, the disease or disorder is responsive to the inhibition of the GIRK1/4 receptor.

In yet another aspect, the present disclosure relates to a method for maintaining a sinus rhythm after cardioversion in a patient with persistent or recent onset of atrial fibrillation or preventing a recurrence in a patient with paroxysmal atrial fibrillation, the method comprising administering to a patient in need thereof a therapeutically effective amount of a compound of the present disclosure, or a pharmaceutically acceptable salt thereof, or a pharmaceutical composition comprising the compound of the disclosure, or a pharmaceutically acceptable salt thereof, and a pharmaceutically acceptable carrier.

In another aspect. the present disclosure relates to a compound of the disclosure, or a pharmaceutically acceptable salt thereof, for use a medicament.

Another aspect of the present disclosure relates to a compound of the disclosure, or a pharmaceutically acceptable salt thereof, or a pharmaceutical composition comprising the compound of the disclosure, or a pharmaceutically acceptable salt thereof, and a pharmaceutically acceptable carrier for use in the in the treatment of a disease or disorder responsive to the inhibition of the GIRK1/4 receptor.

In another aspect, the present disclosure relates to a compound of the disclosure, or a pharmaceutically acceptable salt thereof, or a pharmaceutical composition comprising the compound of the disclosure, or a pharmaceutically acceptable salt, thereof, and a pharmaceutically acceptable carrier for use in the treatment, prevention, inhibition, or elimination of a disease or disorder, wherein the disease or disorder is selected from cardiac arrhythmia, atrial fibrillation, bradyarrhythmia, bradycardia, heart block, sick sinus syndrome, parasympathetic hyperactivation, primary hyperaldosteronism, hypotension, and vasovagal syncope.

In another aspect, the present disclosure relates to a compound of the disclosure, or a pharmaceutically acceptable salt thereof, or a pharmaceutical composition comprising the compound of the disclosure, or a pharmaceutically acceptable salt, thereof, and a pharmaceutically acceptable carrier for use in maintaining a sinus rhythm after cardioversion in a patient with persistent or recent onset of atrial fibrillation or preventing a recurrence in a patient with paroxysmal atrial fibrillation.

Another aspect of the present disclosure relates to the use of a compound of the disclosure, or a pharmaceutically acceptable salt thereof, or a pharmaceutical composition comprising the compound of the disclosure, or a pharmaceutically acceptable salt thereof, and a pharmaceutically acceptable carrier in the manufacture of a medicament treating a disease or disorder responsive to the inhibition of the GIRK1/4 receptor.

In another aspect, the present disclosure relates to the use of a compound of the disclosure, or a pharmaceutically acceptable salt thereof, or a pharmaceutical composition comprising the compound of the disclosure, or a pharmaceutically acceptable salt thereof, and a pharmaceutically acceptable carrier in the treatment of a disease or disorder, wherein the disease or disorder is selected from cardiac arrhythmia, atrial fibrillation, bradyarrhythmia, bradycardia, heart block, sick sinus syndrome, parasympathetic hyperactivation, primary hyperaldosteronism, hypotension, and vasovagal syncope.

In another aspect, the present disclosure relates to the use of a compound of the disclosure, or a pharmaceutically acceptable salt thereof, or a pharmaceutical composition comprising the compound of the disclosure, or a pharmaceutically acceptable salt thereof, and a pharmaceutically acceptable carrier in maintaining a sinus rhythm after cardioversion in a patient with persistent or recent onset of atrial fibrillation or preventing a recurrence in a patient with paroxysmal atrial fibrillation.

In certain embodiments, the disease or disorder is a disease or disorder responsive to the inhibition of the GIRK1/4 receptor. In some embodiments, the disease or disorder responsive to the inhibition of the GIRK1/4 receptor is selected from cardiac arrhythmia, atrial fibrillation, bradyarrhythmia, bradycardia, heart block, sick sinus syndrome, parasympathetic hyperactivation, primary hyperaldosteronism, hypotension, and vasovagal syncope.

In some embodiments, the disease or disorder is selected from cardiac arrhythmia, atrial fibrillation, bradyarrhythmia, bradycardia, heart block, sick sinus syndrome, parasympathetic hyperactivation, primary hyperaldosteronism, hypotension, and vasovagal syncope.

The disclosed compounds of the disclosure can be administered in effective amounts to treat or prevent a disorder and/or prevent the development thereof in subjects.

In some embodiments, administering the compound is orally.

### Combination Therapy

The compounds of the present disclosure can be administered in therapeutically effective amounts in a combinational therapy with one or more pharmaceutically active agents (pharmaceutical combinations) or modalities, e.g., non-drug therapies. For example, synergistic effects can occur with other cardiovascular agents, antihypertensive agents, coronary vasodilators, and diuretic substances. Where the compounds of the application are administered in conjunction with other therapies, dosages of the co-administered compounds will of course vary depending on the type of co-drug employed, on the specific drug employed, on the condition being treated and so forth.

The compounds of the present disclosure may be administered either simultaneously with, or before or after, one or more other pharmaceutically active agent. The compound of the present disclosure may be administered separately, by the same or different route of administration, or together in the same pharmaceutical composition as the other agents. A pharmaceutically active agent is, for example, a chemical compound, peptide, antibody, antibody fragment or nucleic acid, which is therapeutically active or enhances the therapeutic activity when administered to a patient in combination with a compound of the present disclosure.

In one embodiment, the disclosure provides a product comprising a compound of the present disclosure and at least one other pharmaceutically active agent as a combined preparation for simultaneous, separate or sequential use in therapy. In one embodiment, the therapy is the treatment of is a disease or disorder responsive to the inhibition of the GIRK1/4 receptor. Products provided as a combined preparation include a composition comprising the compound of the present disclosure and the other pharmaceutically active agent(s) together in the same pharmaceutical composition as described herein, or the compound of the present disclosure and the other pharmaceutically active agent(s) in separate form, e.g., in the form of a kit.

In another aspect, the disclosure includes a compound of the present disclosure, or a pharmaceutically acceptable salt thereof, for use in a combination therapy.

Another aspect of the disclosure is directed to pharmaceutical compositions comprising a compound of the disclosure, or a pharmaceutically acceptable salt thereof, a pharmaceutically acceptable carrier, and one or more pharmaceutically active agent. The pharmaceutical acceptable carrier may further include an excipient, diluent, or surfactant.

Combination therapy includes the administration of the subject compounds in further combination with other biologically active ingredients. For instance, the compounds of the present disclosure can be used in combination with other pharmaceutically active agents, preferably compounds that are able to enhance the effect of the compounds of the application. The compounds of the present disclosure can be administered simultaneously (as a single preparation or separate preparation) or sequentially to the other drug therapy or treatment modality. In general, a combination therapy envisions administration of two or more drugs during a single cycle or course of therapy.

Exemplary additional pharmaceutically active agents that may be used in combination with the compounds of the present disclosure, include, but are not limited to, any other antiarrhythmic agent, such as Class I antiarrhythmic agents (e.g., quinidine, lidocaine, and propafenone). Class II antiarrhythmic agents (e.g., propranolol), Class III antiarrhythmic agents (e.g., sotalol, dofetilide, amiodarone, dronedarone, budiodarone, azimilide and ibutilide), Class IV antiarrhythmic agents (e.g., diltiazem and verapamil), Class V antiarrhythmic agents (e.g., adenosine), cardiac glycosides (e.g., digitalis and ouabain) and other drugs affecting atrial refractoriness (e.g. I_{Na,Late} blockers such as described in WO 2013/112932); haemostasis modulators, including antithrombotics such as activators of fibrinolysis; thrombin inhibitors; factor VIIa inhibitors; anticoagulants, such as vitamin K antagonists (e.g., warfarin), heparin and low molecular weight analogues thereof (e.g., dalteparin), factor Xa inhibitors (e.g., rivaroxaban and apixaban), and direct thrombin inhibitors (e.g., argatroban); antiplatelet agents, such as cyclooxygenase inhibitors (e.g., aspirin and NSAIDs), adenosine diphosphate (ADP) receptor inhibitors (e.g., clopidogrel), phosphodiesterase inhibitors (e.g., cilostazol), glycoprotein IIB/IIA inhibitors (e.g., tirofiban), and adenosine reuptake inhibitors (e.g., dipyridamole), anti-dyslipidemia agents, such as HMG-COA reductase inhibitors (statins) and other cholesterol-lowering agents; PPARa agonists (fibrates, e.g., gemfibrozil and fenofibrate); bile acid sequestrants (e.g., cholestyramine); cholesterol absorption inhibitors (e.g., plant sterols (i.e., phytosterols), synthetic inhibitors); cholesteryl ester transfer protein (CETP) inhibitors; inhibitors of the ileal bile acid transport system (IBAT inhibitors); bile acid binding resins; nicotinic acid (niacin) and analogues thereof; anti-oxidants; and omega-3 fatty acids; antihypertensive agents, including adrenergic receptor antagonists, such as beta blockers (e.g., atenolol), alpha blockers (e.g., doxazosin), and mixed alpha/beta blockers (e.g., labetalol); adrenergic receptor agonists, including alpha-2 agonists (e.g., clonidine); angiotensin converting enzyme (ACE) inhibitors (e.g., lisinopril), calcium channel blockers, such as dihydropyridines (e.g., nifedipine), phenylalkylamines (e.g., verapamil), and benzothiazepines (e.g., diltiazem); angiotensin II receptor antagonists (e.g., losartan); aldosterone receptor antagonists (e.g., eplerenone); centrally acting adrenergic drugs, such as central alpha agonists (e.g., clonidine); and diuretic agents (e.g., furosemide); anti-obesity agents, such as appetite suppressant (e.g., ephedrine), including noradrenergic agents (e.g., phentermine) and serotonergic agents (e.g., sibutramine), pancreatic lipase inhibitors (e.g., orlistat), microsomal transfer protein (MTP) modulators, diacyl glycerolacyltransferase (DGAT) inhibitors, and cannabinoid (CBI) receptor antagonists (e.g., rimonabant); insulin and insulin analogues; insulin secretagogues, including sulphonylureas (e.g., glipizide) and prandial glucose regulators (sometimes called “short-acting secretagogues”), such as meglitinides (e.g., repaglinide and nateglinide); agents that improve incretin action, for example dipeptidyl peptidase IV (DPP-4) inhibitors (e.g., vildagliptin, sitagliptin, LAF237. MK-431), and glucagon-like peptide-I (GLP-1) agonists (e.g., exenatide); insulin sensitising agents including peroxisome proliferator activated receptor gamma (PPARy) agonists, such as thiazolidinediones (e.g., pioglitazone and rosiglitazone), and agents with any combination of PPAR alpha, gamma and delta activity; agents that modulate hepatic glucose balance, for example biguanides (e.g., metformin), fructose 1,6-bisphosphatase inhibitors, glycogen phopsphorylase inhibitors, glycogen synthase kinase inhibitors, and glucokinase activators; agents designed to reduce/slow the absorption of glucose from the intestine, such as alpha-glucosidase inhibitors (e.g., miglitol and acarbose); agents which antagonise the actions of or reduce secretion of glucagon, such as amylin analogues (e.g., pramlintide); agents that prevent the reabsorption of glucose by the kidney, such as sodium-dependent glucose transporter 2 (SGLT-2) inhibitors.

The term “HMG-Co-A reductase inhibitor” (also called beta-hydroxy-beta-methylglutaryl-co-enzyme-A reductase inhibitors) includes active agents that may be used to lower the lipid levels including cholesterol in blood. Examples include atorvastatin, cerivastatin, compactin, dalvastatin. dihydrocompactin, fluindostatin, fluvastatin, lovastatin, pitavastatin, mevastatin, pravastatin, rivastatin, simvastatin, and velostatin, or pharmaceutically acceptable salts thereof.

The term “ACE-inhibitor” (also called angiotensin converting enzyme inhibitors) includes molecules that interrupt the enzymatic degradation of angiotensin I to angiotensin II. Such compounds may be used for the regulation of blood pressure and for the treatment of congestive heart failure. Examples include alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, pharmaceutically acceptables salt thereof.

An angiotensin II receptor antagonist or a pharmaceutically acceptable salt thereof is understood to be an active ingredient which bind to the ATI-receptor subtype of angiotensin II receptor but do not result in activation of the receptor. As a consequence of the inhibition of the AT₁ receptor, these antagonists can, for example, be employed as antihypertensives or for treating congestive heart failure.

The term “diuretic” includes thiazide derivatives (e.g., chlorothiazide, hydrochlorothiazide, methylclothiazide, and chlorothalidon).

DPP-IV is responsible for inactivating GLP-1. More particularly, DPP-IV generates a GLP-1receptor antagonist and thereby shortens the physiological response to GLP-1. GLP-1 is a major stimulator of pancreatic insulin secretion and has direct beneficial effects on glucose disposal. The DPP-IV inhibitor can be peptidic or, preferably, non-peptidic. DPP-IV inhibitors include but are not limited to sitagliptin, linagliptin, saxagliptin, and alogliptin. DPP-IV inhibitors are also generically and specifically disclosed e.g., in WO 98/19998, DE 196 16 486 A1, WO 00/34241 and WO 95/15309, in each case in particular in the compound claims and the final products of the working examples, the subject-matter of the final products, the pharmaceutical preparations and the claims are hereby incorporated into the present application by reference to these publications. Preferred are those compounds that are specifically disclosed in Example 3 of WO 98/19998 and Example 1 of WO 00/34241, respectively.

GLP-1 is an insulinotropic protein which is described, e.g., by W. E. Schmidt et al. in Diabetologia, 28, 1985, 704-707 and in U.S. Pat. No. 5,705,483. The term “GLP-1 agonists” includes variants and analogs of GLP-1 (7-36) NH: which are disclosed in particular in U.S. Pat. Nos. 5,120,712, 5,118,666, 5,512,549, WO 91/11457 and by C. Orskov et al in J. Biol. Chem. 264 (1989) 12826. Further examples include GLP-1 (7-37), in which compound the carboxy-terminal amide functionality of Arg³⁶ is displaced with Gly at the 37^{th} position of the GLP-1(7-36) NH₂ molecule and variants and analogs thereof including GLN⁹-GLP-1(7-37), D-GLN⁹-GLP-J(7-37), acetyl LYS⁹-GLP-1(7-37), LYS¹⁸-GLP-1(7-37) and, in particular. GLP-1(7-37)OH, VAL⁸-GLP-1(7-37), GLYS-GLP-1(7-37), THRS-GLP-1(7-37), MET⁸-GLP1(7-37) and 4-imidazopropionyl-GLP-1, GLP-1 receptor agonists include, but are not limited to, semaglutide, exenatide, and liraglutide.

An aldosterone synthase inhibitor or a pharmaceutically acceptable salt thereof is understood to be an active ingredient that has the property to inhibit the production of aldosterone. Aldosterone synthase (CYP11B2) is a mitochondrial cytochrome P450 enzyme catalyzing the last step of aldosterone production in the adrenal cortex, i.e., the conversion of 11-deoxycorticosterone to aldosterone. The inhibition of the aldosterone production with so-called aldosterone synthase inhibitors is known to be a successful variant to treatment of hypokalemia, hypertension, congestive heart failure, atrial fibrillation or renal failure. Such aldosterone synthase inhibition activity is readily determined by those skilled in the art according to standard assays (e.g., US 2007/0049616).

The class of aldosterone synthase inhibitors comprises both steroidal and non-steroidal aldosterone synthase inhibitors, the latter being most preferred.

The class of aldosterone synthase inhibitors comprises compounds having differing structural features. For example, mention may be made of the compounds which are selected from the group consisting of the non-steroidal aromatase inhibitors anastrozole, fadrozole (including the (+)-enantiomer thereof), as well as the steroidal aromatase inhibitor exemestane, or, in each case where applicable, a pharmaceutically acceptable salt thereof.

The most preferred non-steroidal aldosterone synthase inhibitor is the (+)-enantiomer of the hydrochloride of fadrozole.

A preferred steroidal aldosterone antagonist is eplerenone or Spironolactone; or, in each case, if appropriable, a pharmaceutically acceptable salt thereof.

Aldosterone synthase inhibitors useful in said combination are compounds and analogs generically and specifically disclosed e.g., in US 2007/0049616, in particular in the compound claims and the final products of the working examples, the subject-matter of the final products, the pharmaceutical preparations and the claims are hereby incorporated into the present application by reference to this publication.

The term aldosterone synthase inhibitors also include compounds and analogs disclosed in WO 2008/076860, WO 2008/076336, WO 2008/076862, WO 2008/027284, WO 2004/046145, WO 2004/014914, WO 2001/076574.

Furthermore, aldosterone synthase inhibitors also include compounds and analogs disclosed in US 2007/0225232, US 2007/0208035, US 2008/0318978, US 2008/0076794, US 2009/0012068. US 2009/0048241 as well as in WO 2006/005726, WO 2006/128853, WO 2006128851, WO 2006/128852, WO 2007/065942, WO 2007/116099, WO 2007/116908, WO 2008/119744, and in EP 1886695.

The term “CETP inhibitor” refers to a compound that inhibits the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). Examples include compounds disclosed in U.S. Pat. Nos. 6,140,343 and 6,197,786 (e.g., [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib); compounds disclosed in U.S. Pat. No. 6,723,752 (e.g., (2R)-3-{[3-(4-Chloro-3-ethyl-phenoxy)-phenyl]-[[3-(1,1,2,2-tetrafluoro-ethoxy)-phenyl]-methyl]-amino}-1,1,1-trifluoro-2-propanol); compounds disclosed in U.S. patent application Ser. No. 10/807,838; polypeptide derivatives disclosed in U.S. Pat. No. 5,512,548; rosenonolactone derivatives and phosphate-containing analogs of cholesteryl ester disclosed in J. Antibiot. 49 (8): 815-816 (1996), and Bioorg. Med. Chem. Lett.:; 6:1951-1954 (1996), respectively. Furthermore, the CETP inhibitors also include those disclosed in WO 2000/017165, WO 2005/095409 and WO 2005/097806.

In another embodiment, the other therapeutic agent is selected from any other antiarrhythmic agent, such as Class I antiarrhythmic agents (e.g., quinidine, lidocaine, and propafenone). Class II antiarrhythmic agents (e.g., propranolol), Class III antiarrhythmic agents (e.g., sotalol, dofetilide, amiodarone, dronedarone, budiodarone, azimilide and ibutilide), Class IV antiarrhythmic agents (e.g., diltiazem and verapamil), Class V antiarrhythmic agents (e.g., adenosine), cardiac glycosides (e.g., digitalis and ouabain) and other drugs affecting atrial refractoriness (e.g., I_{Na,Late} blockers such as described in WO 2013/112932).

“Combination therapy” is intended to embrace administration of these therapeutic agents in a sequential manner, wherein each therapeutic agent is administered at a different time and in any order, or in alternation and in any order, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

### Methods for the Preparation of Compounds of Formula (I)

The compounds of the present disclosure may be made by a variety of methods, including standard chemistry. Suitable synthetic routes are depicted in the Schemes given below.

The compounds of the present disclosure may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of the present disclosure.

Those skilled in the art will recognize if a stereocenter exists in the compounds of the present disclosure. Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Definitions

Terms not specifically defined herein should be given the meanings that would be given to them by one of skill in the art in light of the disclosure and the context. For purposes of interpreting this specification, the following definitions will apply unless specified otherwise and whenever appropriate, terms used in the singular will also include the plural and vice versa.

It must be noted that as used herein and in the appended claims, the singular forms “a”, “an” and “the” include plural referents unless the context clearly dictates otherwise. Thus, for example, reference to “the pharmaceutical formulation” includes reference to one or more pharmaceutical formulations; and so forth.

As used herein, the term “phosphoric acid ester derivative” refers to a derivative of the compound of formula (I) as defined herein wherein at least one free —OH group is converted into a phosphoric acid ester group.

The terms “compound of formula (I)” and “parent compound” are used berein interchangeably to mean a compound as defined herein free from phosphoric acid ester groups. The term “compound of formula (I)”, as used herein, means a compound as defined herein formed after cleavage upon hydrolysis of the phosphoric acid ester group, and is used interchangeably with the term “parent compound”.

The term “acyl”, as used herein, refers to a group represented by the general formula hydrocarbylC(O)—, preferably alkylC(O)—.

The term “acyloxy”, as used herein, refers to a group represented by the general formula hydrocarbylC(O)O—, preferably alkylC(O)O—.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated. Examples of alkenyl groups include ethenyl, propenyl, n-butenyl, iso-butenyl, pentenyl, or hexenyl.

The term “alkoxy”, as used herein, refers to an alkyl group, preferably a lower alkyl group, having an oxygen attached thereto, e.g., —O(alkyl). Representative alkoxy groups include, but are not limited to, methoxy, ethoxy, propoxy, tert-butoxy and the like. Representative substituted alkoxy groups include, but are not limited to, —OCF₃ and the like.

An “alkyl” group or “alkane” is a straight chained or branched non-aromatic hydrocarbon which is completely saturated. Typically, a straight chamed or branched alkyl group has from 1 to about 20 carbon atoms, preferably from 1 to about 10 unless otherwise defined. Examples of straight chained and branched alkyl groups include methyl ethyl, n-propyl, iso-propyl, o-butyl, sec-butyl, tert-butyl, pentyl, hexyl, pentyl and octyl. A C₁-C₆ straight chained or branched alkyl group is also referred to as a “lower alkyl” group. An “alkylene” group is a divalent straight chained or branched non-aromatic hydrocarbon which is completely saturated. Typically a divalent straight chamed or branched alkylene group has from 1 to about 20 carbon atoms, preferably from 1 to about 10 unless otherwise defined. Examples of divalent straight chained and branched alky lene groups include methylene, ethylene, n-propylene, iso-propylene, n-buty lene, sec-buty lene, tert-buty lene, pentylene, hexylene, penty lene and octylene.

The term “alkynyl”, as used herein, refers to an aliphatic group containing at least one triple bond. Examples of alkynyl groups include ethynyl, propargyl, n-butynyl, iso-butynyl, pentynyl, or hexynyl.

The term “aryl”, as used herein, include substituted or unsubstituted single-ring aromatic groups in which each atom of the ring is carbon. Preferably the ring is a 5-to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described herein, but that contain at least one double or triple bond respectively.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo [4.2.0]oct-3-ene, naphthalene and adamantane. Exemplary fused carbocycles include decalin, naphthalene, 1,2,3,4-tetrahydronaphthalene, bicyclo [4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms. more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings.

The terms “halo” and “halogen”, as used herein, means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polyeyelic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, but are not limited to, furyl, thienyl, pyrrolyl, pyridyl, pyridyl N-oxide, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo [1,2-b]pyrazolyl, furo[2,3-c]pyridinyl, imidazo [1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b] pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido [4,3-b][1,6] naphthyridinyl, thieno[2,3-b]pyrazinyl, quinazolinyl, tetrazolo [1,5-a]pyridinyl, [1,2,4]triazolo [4,3-a]pyridinyl, isoindolyl, pyrrolo[2,3-b]pyridinyl, pyrrolo[3,4-b]pyridinyl, pyrrolo [3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1Δ2-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4 d]thiazolyl, imidazo[2,1-b][1,3,4]thiadiazolyl, thieno[2,3-b]pyrrolyl, 3H-indolyl, indolinyl, indolinonyl, dihydrobenzothiophenyl, dihydrobenzofuran, chromanyl, thiochromanyl, tetrahydroquinolinyl, dihydrobenzothiazine,3,4-dihydro-1H-isoquinolinyl, 2,3-dihydrobenzofuran, indolinyl, indolyl, and dihydrobenzoxanyl.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3-to 10-membered rings, more preferably 3-to 7-membered rings, whose ring structures include at least one heteroatom, preferably one to four heteroatoms, more preferably one or two beteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like. Heterocyclyl groups can also be substituted by oxo groups. For example, “heterocyclyl” encompasses both pyrrolidine and pyrrolidinone.

“Haloalkyl”, as used herein, refers to an alkyl group substituted with one or more halogens. Examples of haloalkyl groups include, but are not limited to, trifluoromethyl, difluoromethyl, pentafluoroethyl, trichloromethyl, etc.

As used herein, the term “oxo” refers to a carbonyl group. When an oxo substituent occurs on an otherwise saturated group, such as with an oxo-substituted cycloalkyl group (e.g., 3-oxo-cyclobutyl), the substituted group is still intended to be a saturated group. When a group is referred to as being substituted by an “oxo” group, this can mean that a carbonyl moicty (i.e., —C(—O)—) replaces a methylene unit (i.e., —CH₂—).

The term “optionally substituted” means that a given chemical moiety (e.g., an alkyl group) can (but is not required to) be bonded to other substituents (e.g., heteroatoms). For instance, an alkyl group that is optionally substituted can be a fully saturated alkyl chain (e.g., a pure hydrocarbon). Alternatively, the same optionally substituted alkyl group can have substituents different from hydrogen, wherein the substituents are as defined herein. “Optionally substituted” as used herein also refers to substituted or unsubstituted whose meaning is described below.

The term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

The term “unsubstituted” means that the specified group bears no substituents.

A “patient” or “subject” is a mammal, e.g., a human, mouse, rat, guinea pig, dog, cat, horse, cow, pig, or nonhuman primate, such as a monkey, chimpanzee, baboon or, rhesus. In certain embodiments, the subject is a primate. In yet other embodiments, the subject is a human.

The terms “pharmaceutically effective amount” or “therapeutically effective amount” or “effective amount” means an amount of a compound according to the present disclosure which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician The amount of a compound according to the present disclosure which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the present disclosure, and the age, body weight, general health, sex, and diet of the patient. Such a therapeutically effective amount can be determined routinely by one of ordinary skill in the art having regard to their own knowledge, the prior art, and this disclosure.

As used herein, the term “pharmaceutical composition” refers to a compound of the present disclosure, or a pharmaceutically acceptable salt, hydrate, solvate, stereoisomer, or tautomer thereof, together with at least one pharmaceutically acceptable carrier, in a form suitable for oral or parenteral administration.

“Carrier” encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

“Combination” refers to either a fixed combination in one dosage unit form, or a combined administration where a compound of the present disclosure and at least one combination partner (e.g., another drug as explained below, also referred to as “therapeutic agent” or “co-agent”) may be administered independently at the same time or separately within time intervals, especially where these time intervals allow that the combination partners show a beneficial effect from the co-action of these therapeutic agents. The beneficial effect of the combination includes, but is not limited to, a cooperative, e.g., synergistic, effect and/or a pharmacokinetic or pharmacodynamic co-action, or any combination thereof, resulting from the combination of therapeutic agents. In one embodiment, administration of these therapeutic agents in combination is carried out over a defined time period (e.g., minutes, hours, days or weeks depending upon the combination selected).

The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time.

The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., a compound of the present disclosure and a combination partner, are both administered to a patient simultaneously in the form of a single entity or dosage. The term “non-fixed combination” means that the therapeutic agents, e.g., a compound of the present disclosure and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agents.

A subject is “in need of” a treatment if such subject would benefit biologically, medically, or in quality of life from such treatment (preferably, a human).

As used herein, the term “inhibit”, “inhibition”, or “inhibiting” refers to the reduction or suppression of a given condition, symptom, or disorder, or disease, or a significant decrease in the baseline activity of a biological activity or process.

As used herein, the term “treat”, “treating”, or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing”, or “prevention” of any disease or disorder refers to the prophylactic treatment of the disease or disorder; or delaying the onset or progression of the disease or disorder.

“Pharmaceutically acceptable” means that the substance or composition must be compatible chemically and/or toxicologically, with the other ingredients comprising a formulation, and/or the mammal being treated therewith.

“Disorder” means, and is used interchangeably with, the terms discase, condition, or illness, unless othenvise indicated.

“Administer”, “administering”, or “administration” means to either directly administering a disclosed compound or pharmaceutically acceptable salt of the disclosed compound or a composition to a subject, or administering a pro-drug derivative or analog of the compound or pharmaceutically acceptable salt of the compound or composition to the subject, which can form an equivalent amount of active compound within the subject's body.

“Compounds of the present disclosure”, “compounds of the disclosure”, and equivalent expressions (unless specifically identified otherwise) refer to phosphoric acid ester derivatives of the compounds of formula (I) as well as compounds of any of formulae (II), (III), (III-A), (III-B), (IV), (IV-A) or (V) as defined herein including the salts, particularly the pharmaceutically acceptable salts thereof, where the context so permits thereof, as well as all stereoisomers (including diastereoisomers and enantiomers), rotamers, tautomers, and isotopically labelled compounds (including deuterium (“D”) substitutions).

In a specific embodiment, the term “about” or “approximately” means within 20%, preferably within 10%, and more preferably within 5% of a given value or range.

As used herein, “disorders or diseases responsive to the inhibition of the GIRK1/4 receptor” and “disorders responsive to the inhibition of the GIRK1/4 receptor,” and like terms include, but are not limited to, cardiac arrhythmia, atrial fibrillation, bradyarrhythmia, bradycardia, heart block, sick sinus syndrome, parasympathetic hyperactivation, primary hyperaldosteronism, hypotension, and vasovagal syncope.

### EXAMPLES

The present disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (1999) Protective Groups in Organic Synthesis. 3rd edition. John Wiley & Sons). These groups are removed at a convenient stage of the compound synthesis using methods that are readily apparent to those skilled in the art.

Unless otherwise noted, reagents and solvents were used as received from commercial suppliers. Proton nuclear magnetic resonance (NMR) spectra were obtained on either Bruker Avance spectrometer or Varian Oxford 400 MHz spectrometer unless otherwise noted. NMR spectra are given in ppm (δ) and coupling constants, J, are reported in Hertz. Tetramethylsilane (TMS) was used as an internal standard. Chemical shifts are reported in ppm relative to dimethyl sulfoxide (δ 2.50). methanol (δ 3.31), chloroform (δ 7.26) or other solvent as indicated in NMR spectral data. A small amount of dry sample (2-5 mg) is dissolved in an appropriate deuterated solvent (1 mL). Mass spectra (ESI-MS) were collected using a Waters System (Acquity UPLC and a Micromass ZQ mass spectrometer) or Agilent-1260 Infinity (6120 Quadrupole); all masses reported are the m/z of the protonated parent ions unless recorded otherwise. The chemical names were generated using ChemBioDraw Ultra v14 from CambridgeSoft.

Temperatures are given in degrees Celsius. As used herein, unless specified otherwise, the term “room temperature” or “ambient temperature” means a temperature of from 15 degrees centigrade to 30 degrees centigrade, such as of from 20 degrees centigrade to 30 degrees centigrade, such as of from 20 degrees centigrade to 25 degrees centigrade. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present disclosure are either commercially available or can be produced by organic synthesis methods known to one of ordinary skill in the art.

TABLE 1
Abbreviations used in the following examples and elsewhere herein are: aqueous

| | | | |
|---|---|---|---|
| ACN | acetonitrile | aq. | aqueous |
| 9-BBN | 9-borabicyclo[3.3.1]nonane | BOC | tert-butyloxycarbonyl |
| br | broad | bs | broad singlet |
| ° C. | degrees celsius | conc. | concentrated |
| δ | NMR chemical shift in ppm | d | doublet |
| | downfield from tetramethylsilane | | |
| DBU | 1,8-diazabicyclo[5.4.0]undec-7-ene | DCM | dichloromethane |
| DIAD | diisopropyl azodicarboxylate | DMAP | 4-(dimethylamino)pyridine |
| DME | dimethoxyethane | DMF | N,N-dimethylformamide |
| DMSO | dimethylsulfoxide | DPPF | 1,1′- |
| | | | bis(diphenylphosphino)ferrocene |
| eq. | equivalent | Et | ethyl |
| EtOAc | ethyl acetate | g | gram |
| HEPES | (4-(2-hydroxyethyl)-1- | h(r) | hour |
| | piperazineethanesulfonic acid | | |
| L | liter | i-Pr | isopropyl |
| m | multiplet | LC/MS | liquid chromatography-mass |
| | | | spectrometry |
| mg | milligram | M | molarity |
| min | minute | Me | methyl |
| μL | microliter | MHz | megahertz |
| mmol | millimole | mL | milliliter |
| n-Bu | normal butyl | N | normal |
| NMR | nuclear magnetic resonance | n-BuLi | n-butyllithium |
| Ph | phenyl | NMP | N-methyl-2-pyrrolidone |
| ppm | parts per million | pH | −log₁₀H⁺ concentration |
| Rt | retention time | q | quartet |
| s | singlet | RP-HPLC | reverse-phase high performance |
| | | | liquid chromatography |
| t | triplet | sat. | saturated |
| temp. | temperature | t-Bu | tert-butyl |
| THF | tetrahydrofuran | TFA | trifluoroacetic acid |
| wt | weight | | |

### General Synthetic Schemes

Compounds of formula (II) of the present disclosure can be prepared as described below in Scheme A by reaction of a primary alcohol of the compounds of formula (I), wherein L is (C₁-C₆) alkylene optionally substituted with one or more —OH and A and R³ are as defined herein, with a suitable phosphorylation reagent (e.g., tetrabenzyl diphosphate) to provide an intermediate compound of formula (I-1) with the so formed phosphorylated alcohol group, wherein P¹ is a suitable protecting group, and subsequent dephosphorylation to provide the desired phosphoric acid ester compounds of formula (II).

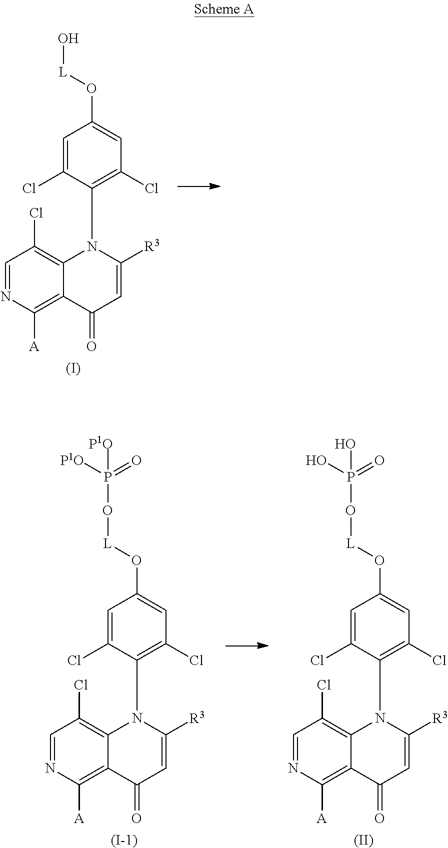

Alternatively, compounds of formula (IV-1) of the present disclosure with a primary and a secondary alcohol function, wherein L, R^{d} and R³ as as defined herein, can be utilized with the primary alcohol function protected with a suitable protecting group (P²) as described below in Scheme B. As shown in Scheme B, the secondary alcohol is reacted with a suitable phosphorylation reagent (e.g., tetrabenzyl diphosphate) and the corresponding intermediate compound of formula (IV-2) is fully deprotected with one or two deprotecting reagents to provide the desired secondary phosphoric acid ester compounds of formula (IV).

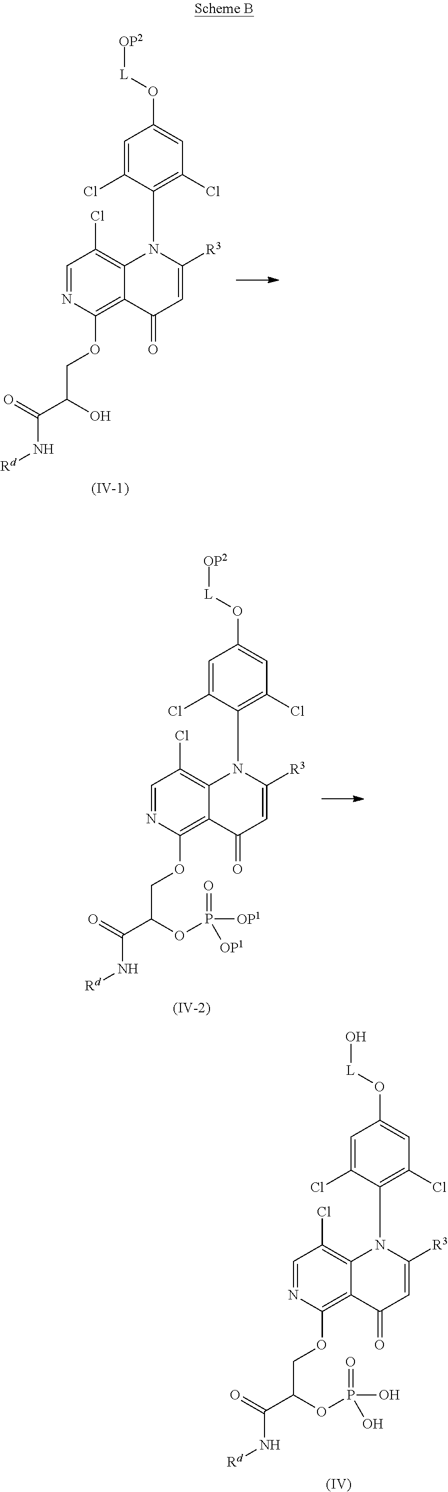

The compounds of formula (I) and derivatives thereof are suitable intermediates for the preparation of the compounds of the present disclosure and can be prepared as described below in any one of Schemes C, D, E and F.

As shown in Scheme C, pyridine A is chlorinated and formylated in a one-pot reaction with POCl₃ in a suitable solvent such as DMF at a suitable temperature, generally from 0° C. to 90° C. (e.g., room temperature), to yield the aromatic aldehyde B. The aldehyde B can be reacted with a deprotonated alkyne (e.g a Grignard reagent), wherein R³ is as defined herein, in a suitable solvent such as THF, at a suitable temperature (e.g., −70° C.) to furnish the benzyl alcohol C. The benzyl alcohol C, wherein R³ is as defined herein, is oxidized to the corresponding ketone under appropriate oxidizing conditions such as the Dess-Martin periodinane reagent in a suitable solvent such as DCM at a suitable temperature, generally from 0° C. to room temperature. Reaction of aniline AX, wherein X is F, Br or I, under Lewis acid-catalysis (e.g. AlCl₃) in a suitable solvent such as DCM at a suitable temperature, generally from 0° C. to room temperature, provides intermediates E, wherein X is F, Bror I and R³ is as defined herein. The intermediates E can be cyclized to the annealated 4-pyridones F, wherein X is F, Br or I and R³ is as defined herein, under basic conditions (e.g. with triethylamine in DMF for intermediates E, wherein X is For I and R³ is as defined herein, or with NaOH in DCM for intermediates E, wherein X is Br and R³ is as defined bercin) at a suitable temperature, generally from room temperature to 60° C.

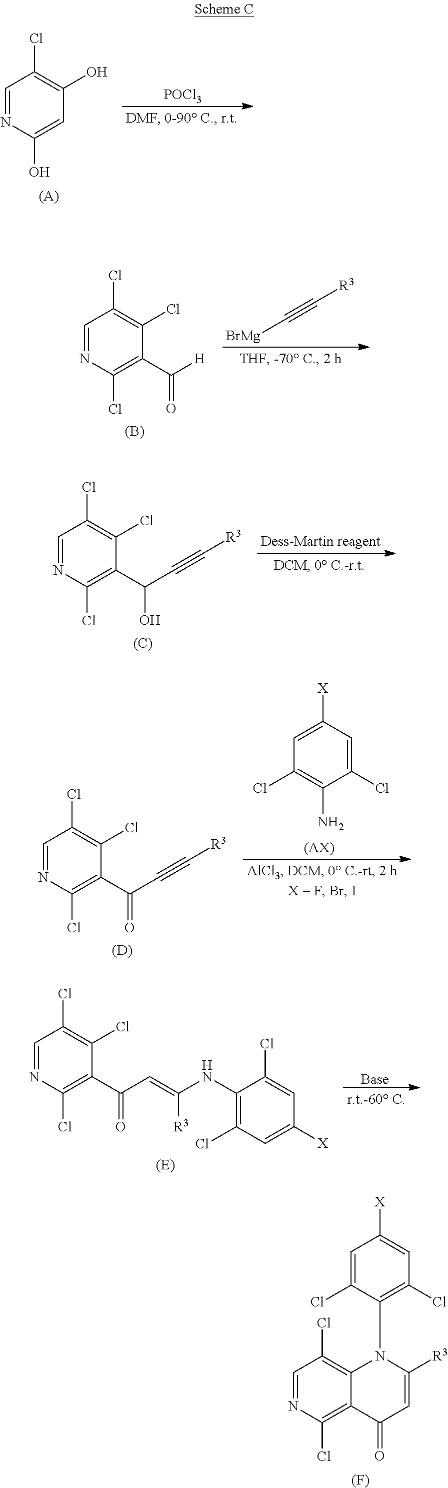

Examples of compounds of the general formula (Ia) can be prepared as described below in Scheme D.

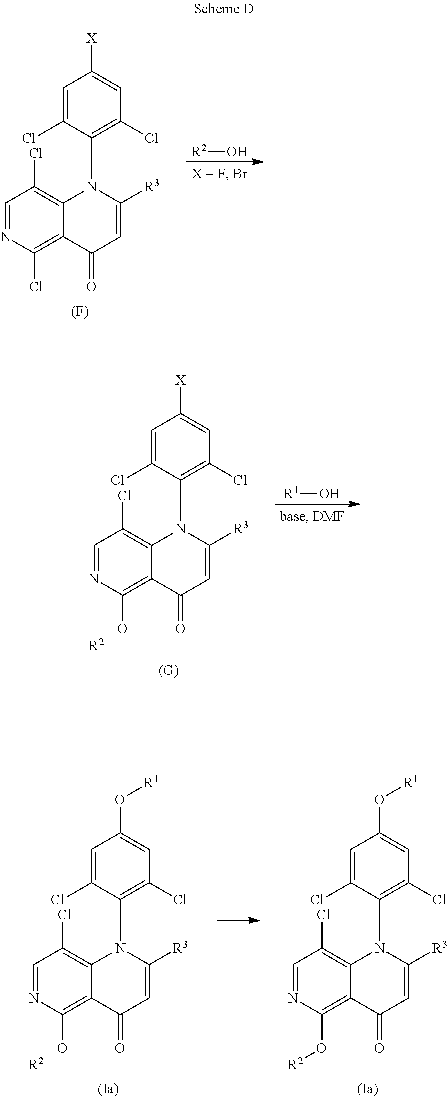

As shown in Scheme D, intermediates F, wherein X is F or Brand R³ is as defined herein, can be converted to intermediates G by nucleophilic aromatic substitution with R²—OH, wherein R² is as defined herein. The substituent-OR1 can be introduced by a second nucleophilic aromatic substitution with R¹—OH, wherein R¹ is (C₁-C₆) alkyl substituted with one or more —OH, using intermediates G, wherein X is F or Br and R² and R³ are as defined herein, yielding compounds of formula (Ia), wherein R¹, R² and R³ are as defined herein. The required R¹—OH and R²—OH alcohols are either commercially available or can be prepared according to literature or in an analogous manner. Additional transformations can be performed. e.g. protecting group manipulations or amide formation anywhere in the synthetic sequence, to yield further compounds of the general formula (Ia), wherein R¹ and R³ are as defined herein, and R″² is (C₁-C₆)alkyl substituted with one or more substituents independently selected from —NHC(O)R^{c} and —C(O)NHR^{d}, wherein the (C₁-C₆) alkyl is further optionally substituted with one or more substituents independently selected from halogen, —OH and —CN, and wherein R² is different from R′².

Alternatively, as shown in Scheme E, intermediates G, wherein X is Br or I and R² and R³ are as defined herein, can be converted to boronate esters H in the presence of a suitable catalyst such as PdCl₂(dppf). The boronate ester H can be oxidized to phenol J, wherein R² and R³ are as defined herein, with hydrogen peroxide in the presence of a suitable catalyst. Intermediates J can be converted via nucleophilic substitution with bromides or triflates in the presence of a suitable base to yield compounds of the general formula (Ia), wherein R¹, R² and R³ are as defined herein. As shown in Scheme B, additional transformations can be performed e.g. protecting group manipulations or amide formation anywhere in the synthetic sequence to yield further compounds of the general formula (Ia), wherein R¹ and R³ are as defined herein, and R′² is (C₁-C₆)alkyl substituted with one or more substituents independently selected from —NHC(O)R^{c} and —C(O)NHR^{d}, wherein the (C₁-C₆) alkyl is further optionally substituted with one or more substituents independently selected from halogen, —OH and —CN, and wherein R² is different from R′².

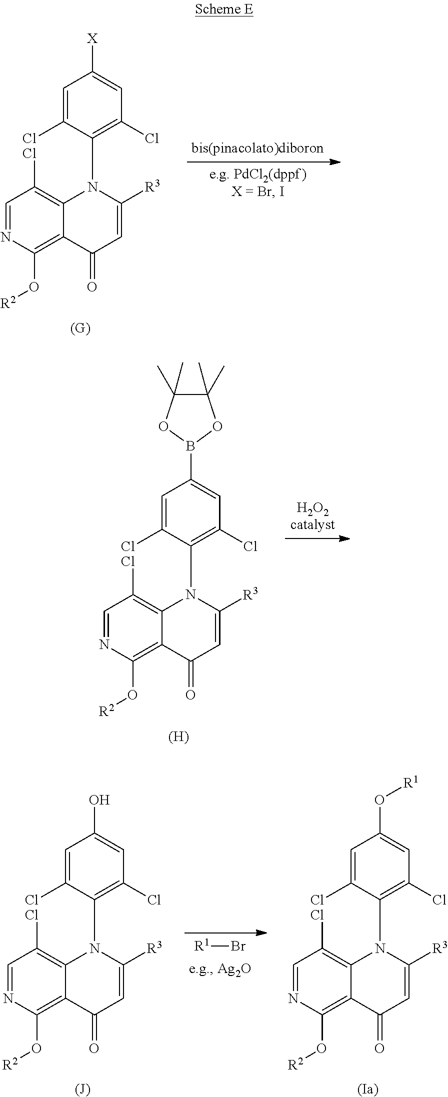

According to Scheme F, intermediates F, wherein X is F, can be also reacted via a Stille coupling with stannanes (e.g., vinyl-or allyl-tributyl stannane) in the presence of a suitable Pd catalyst such as Pd₂ (dba); with a suitable base such as trifurylphosphine to yield compounds K, wherein R_{A} is vinyl or allyl and R³ is as defined herein. Nucleophilic aromatic substitution with R¹—OH, wherein R¹ is as defined herein, leads to intermediates L which can be subjected to addition of sodium alkyl sulfonates R_{A}′SO₂Na, wherein R_{A}′ is (C₁-C₆) alkyl substituted with —SO₂(C₁-C₄)alkyl, in a suitable solvent such as EtOH or AcOH to provide compounds of the general formula (Ib), wherein R_{A}′ is (C₁-C₆) alkyl substituted with —SO₂(C₁-C₄)alkyl and R¹ and R³ are as defined herein. Alternatively, alkenes can be employed to form in situ terminal boranes with 9-BBN which undergo Pd-catalyzed coupling in the presence of a suitable Pd catalyst such as Pd (PPh₃)₄ with intermediates F, wherein X is F, to form intermediates M, wherein R_{B} is (C₁-C₆) alkyl substituted with —S(C₁-C₄)alkyl or —C(O)NHR^{c}, and R³ and R^{c} are as defined herein. In the case of sulfides side chains, oxidation (e.g., with H₂O₂) provides sulfones in a subsequent step. Additional nucleophilic aromatic substitution with R¹—OH, wherein R¹ is as defined herein, provides compounds of the general formula (Ic), wherein R_{B}′ is (C₁-C₆) alkyl substituted with —SO₂(C₁-C₄)alkyl or —C(O)NHR^{b}, and R¹, R³ and R^{b} are as defined herein.

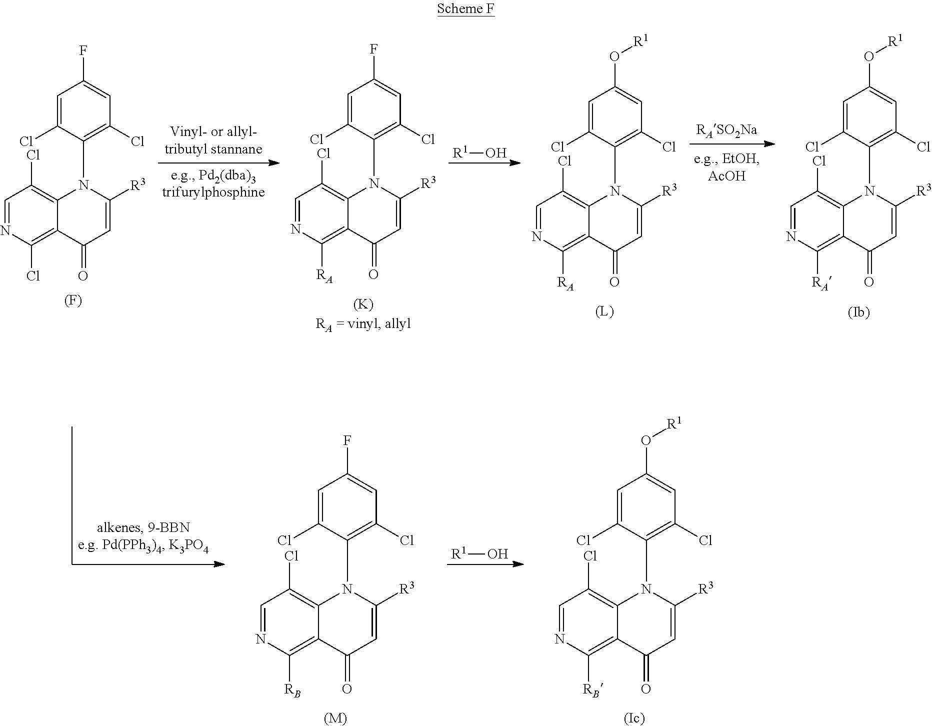

The following LC-MS methods were used for characterization of the examples and intermediates:

### LC/MS Method—Acidic

Pump: Waters AcQuity UPLC
Column: AcQuity UPLC BEH C18 1.7 μM 2.1×30 mm
Column temperature: 50° C.
Gradient (Time/% B): 0.0/2, 0.1/2, 1.5/98, 1.8/98, 1.9/2, 2.0/2
Eluent A: 0.1% Formic acid in water
Eluent B: 0.1% Formic acid in acetonitrile
Flow: 1.0 mL/min
ELSD: SofTA 1100 ELSD
Mass Spec: Waters QDa
LC/MS method-basic
Pump: Waters AcQuity UPLC
Column: AcQuity UPLC BEH C18 1.7 μM 2.1×30 mm
Column temperature: 50° C.
Gradient (Time/% B): 0.0/2, 0.1/2, 1.5/98, 1.8/98, 1.9/2, 2.0/2
Eluent A: 5 mM Ammonium hydroxide in water
Eluent B: 5 mM Ammonium hydroxide acetonitrile
Flow: 1.0 mL/min
ELSD: SofTA 1100 ELSD
Mass Spec: Waters QDa

### Example 1: Synthesis of(S)-2-(3,5-dichloro-4-(8-chloro-5-(2-hydroxy-3-(methylamino)-3-oxopropoxy)-2-methyl-4-oxo-1,6-naphthyridin-1 (4H)-yl)phenoxy)ethyl dihydrogen phosphate

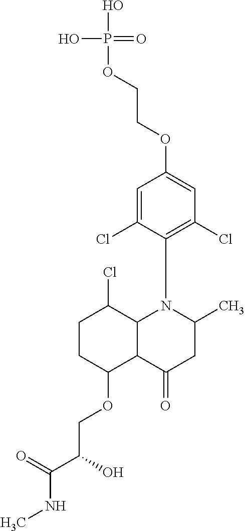

### Step1: Synthesis of(S)-3-((tert-butyldiphenylsilyl)oxy)-2-hydroxy-N-methylpropanamide

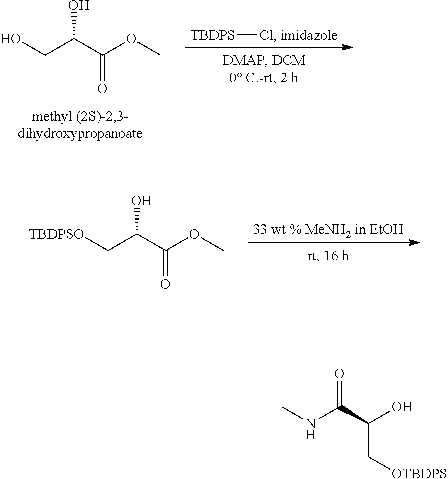

Imidazole (6.263 g. 2.2 eq., 91.99 mmol) and (+)-methyl glycerate (5.022 g, 1 eq., 41.82 mmol) were dissolved in dichloromethane (50 mL) under nitrogen and cooled in an ice bath. DMAP (0.256 g, 0.05 eq., 2.10 mmol) and tert-butyldiphenylchlorosilane (11.58 g. 10.7 mb, 1.008 eq., 42.13 mmol) were added and the mixture was stirred for 10 minutes. A white precipitate formed. The mixture was removed from the cold bath and allowed to stir at ambient temperature. After 2 hours, a sample was evaporated to dryness under reduced pressure and analyzed by NMR. No starting alcohol was found and the reaction was quenched by addition of 50% saturated ammonium chloride (100 mL). The phases were mixed and separated. The organic phase was washed with additional 50% saturated ammonium chloride (100 mL) and the combined aqueous layers were extracted with dichloromethane (50 mL). The combined organic layers were dried with sodium sulfate and evaporated to dryness under reduced pressure. The crude product was treated with methylamine, 33% wt in ethanol (22.7 g. 30.00 mL, 5.78 eq., 242 mmol) under nitrogen. The solution was stirred at ambient temperature ovemight. LC/MS found the intermediate silyl ester had been consumed and the mixture was evaporated to dryness under reduced pressure. The crude yellowish oil was stirred at ambient temperature and heptane (110 mL) was added using an addition funnel over 5 minutes. A white precipitate formed. Once the addition was complete, the suspension was stirred at ambient temperature for 10 minutes and then filtered through a disposable frit. The solids were washed with heptane (2×40 mL) and then dried on the frit to give the desired product as a white solid (11.34 g. 76%). ¹H NMR (400 MHz, CHLOROFORM-d) δppm 1.09 (s, 9H) 2.88 (d, J=5.0 Hz, 3H) 3.87-3.97 (m, 2H) 4.19 (t, J=5.5 Hz, 1H) 6.69-6.86 (br s, 1H) 7.38-7.51 (m, 6H) 7.62-7.68 (m, 4H). m/z: 380.4 [M+Na]⁺.

### Step 2: Synthesis of(S)-3-((tert-butyldiphenylsilyl) oxy)-2-((8-chloro-1-(2,6-dichloro-4-fluorophenyl)-2-methyl-4-oxo-1,4-dihydro-1,6-naphthyridin-5-yl)oxy)-N-methylpropanamide

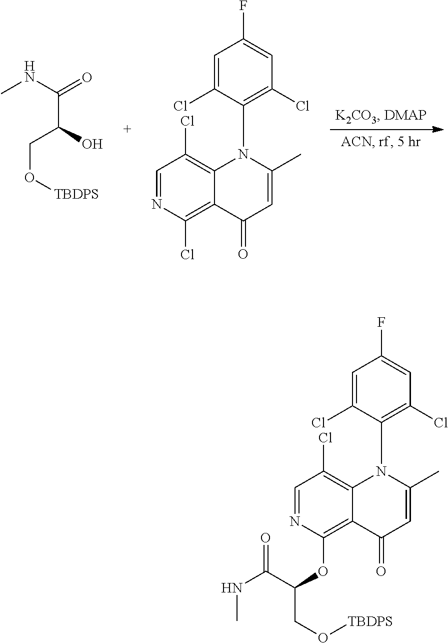

(S)-3-((tert-Butyldiphenylsilyl)oxy)-2-hydroxy-N-methylpropanamide (19.29 g, 1.244 eq., 53.95 mmol), 5,8-dichloro-1-(2,6-dichloro-4-fluorophenyl)-2-methyl-1,6-naphthyridin-4 (1H)-one (17.00 g, 1 eq., 43.36 mmol), vacuum oven dried potassium carbonate (17.98 g. 3 eq., 130.1 mmol), and DMAP (2.119 g, 0.4 eq., 17.35 mmol) were combined in dry acetonitrile (300 mL) under nitrogen. The mixture was heated to gentle reflux under nitrogen. After 5 hours at reflux, the reaction mixture was cooled to ambient temperature. The mixture was filtered through a disposable frit. The solids were washed with acetonitrile and the filtrate was evaporated to dryness under reduced pressure. The crude residue was partitioned between water (300 mL) and ethyl acetate (400 mL). The organic phase was washed with 25% saturated sodium carbonate (300 mL) and dried with brine (200 mL). The combined aqueous layers were extracted with ethyl acetate (2×200 mL). The combined organic layers were washed with 50% saturated ammonium chloride (200 mL) and evaporated to dryness under reduced pressure. The crude was purified by flash chromatography (240 g silica column, 0-50% ethyl acetate in dichloromethane gradient) to give the desired product as a white foam (23.5 g. 76%). m/z: 712.3/714.3 [M+H]⁺.

### Step 3: Synthesis of (S)-3-((tert-butyldiphenylsilyl)oxy)-2-((8-chloro-1-(2,6-dichloro-4-(2-hydroxyethoxy)phenyl)-2-methyl-4-oxo-1,4-dihydro-1,6-naphthyridin-5-yl)oxy)-N-methylpropanamide

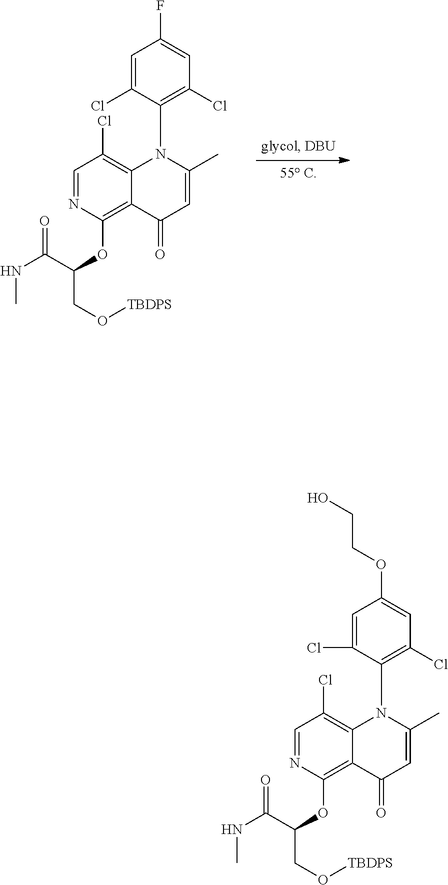

(S)-3-((tert-butyldiphenylsilyl)oxy)-2-((8-chloro-1-(2,6-dichloro-4-fluorophenyl)-2-methyl-4-oxo-1,4-dihydro-1,6-naphthyridin-5-yl)oxy)-N-methylpropanamide (5.80 g, 1 eq., 8.15 mmol), ethylene glycol (dried over molecular sieves, 50.09 g, 45.00 mL, 806.9 mmol) and DBU (10.10 g, 10.00 mL, 66.34 mmol) were combined in a 200 mL round bottom flask under nitrogen and heated in a 70°°C. aluminum block. The reaction was sampled at 30 min, 1 hr, and 90 minutes. The starting material was consumed after 90 minutes and the desired mass was detected as one of the major new peaks. The major impurity appears to be displacement of the N-methyl 2,3-dihydroxypropionamide by glycol. The reaction mixture was cooled to ambient temperature and partitioned between water (250 mL) and ethyl acetate (250 mL). The aqueous phase was extracted with additional ethyl acetate (2×75 mL) and the combined organic layers were dried with sodium sulfate before evaporating to dryness under reduced pressure 6 g crude material (˜85% purity). A small sample of the material was purified by HPLC to yield the desired product. ¹H NMR (400 MHZ, METHANOL-d) δppm 0.97 (s, 9H), 2.09 (s, 3H), 2.93-3.03 (m, 3H), 3.99-4.01 (m, 2H), 4.12-4.23 (m, 2H), 4.26 (dd, J=11.4. 2.2 Hz, 1H), 4.36 (dd, J=11.3, 2.3 Hz, 1H), 5.36-5.48 (m, 1H), 6.59 (s, 1H), 7.11 (t, J=7.6 Hz, 2H), 7.21-7.34 (m, 5H), 7.35-7.41 (m, 1H), 7.43-7.49 (m, 2H), 7.52-7.58 (m, 2H), 7.94 (s, 1H) 9.55-9.35 (m, 1 H). m/z: 754.1/756/1 [M+H]⁺.

### Step 4: Synthesis of (S)-dibenzyl (2-(4-(5-((3-((tert-butyldiphenylsilyl)oxy)-1-(methylamino)-1-oxopropan-2-yl)oxy)-8-chloro-2-methyl-4-oxo-1,6-naphthyridin-1(4H)-yl)-3,5-dichlorophenoxy)ethyl) phosphate.

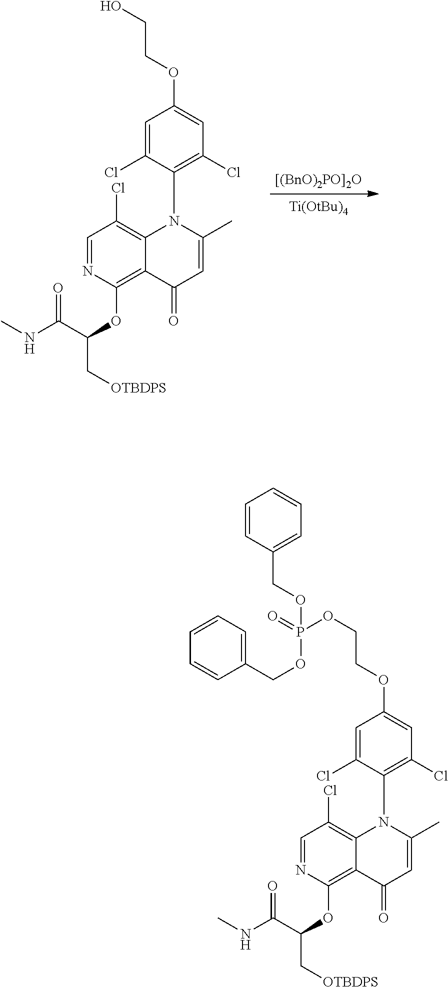

To a solution of (S)-3-((tert-butyldiphenylsilyl)oxy)-2-((8-chloro-1-(2,6-dichloro-4-(2-hydroxyethoxy)phenyl)-2-methyl-4-oxo-1,4-dihydro-1,6-naphthyridin-5-yl) oxy)-N-methylpropanamide (2.0 g, 85% wt. 2.25 mmol) in DCM (20.0 mL) was added tetrabenzyl diphosphate (2.42 g. 2 eq., 4.5 mmol), Hunig's base (873 mg. 1.18 mL, 6.75 mmol) and tetra-tert-butoxytitanium (76.6 mg, 225 μmol). The mixture was stirred at room temperature for 16 hours. Then, tetrabenzyl diphosphate (2.42 g, 2 eq., 4.5 mmol) was added again to the mixture and the mixture was stirred at room temperature for another 24 hours. The mixture was purified by flash chromatography on a silica gel column eluting with 0-100% EtOAc/heptane to give 1.7 g desired product. m/z: 1016.2 [M+H]⁺.

### Step 5: Synthesis of (S)-dibenzyl (2-(3,5-dichloro-4-(8-chloro-5-(2-hydroxy-3-(methylamino)-3-oxopropoxy)-2-methyl-4-oxo-1,6-naphthyridin-1(4H)-yl)phenoxy)ethyl) phosphate.

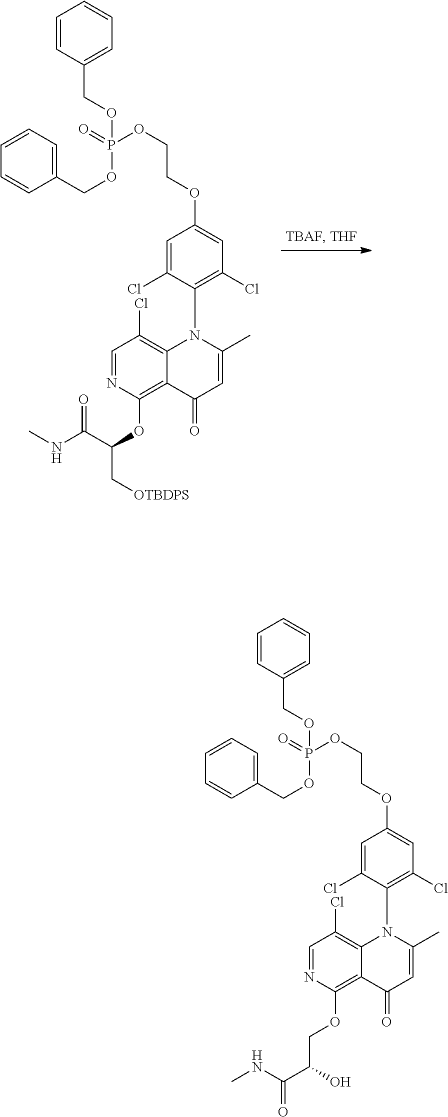

To a solution of (S)-dibenzyl (2-(4-(5-((3-((tert-butyldiphenylsilyl)oxy)-1-(methylamino)-1-oxopropan-2-yl)oxy)-8-chloro-2-methyl-4-oxo-1,6-naphthyridin-1(4H)-yl)-3,5-dichlorophenoxy)ethyl) phosphate (1.7 g, 1.67 mmol) in THF (10.0 mL) was added tetrabutylammonium fluoride (1.0 M in THF, 5.0 mL) at 0°°C. dropwise. The ice bath was removed after addition. The reaction mixture was stirred at 23° C. for 20 minutes until completion of the reaction, as shown by LC/MS analysis. The mixture was diluted with EtOAc and washed with water. The phases were separated and the aqueous layer was extracted with EtOAc twice. The organic layers were combined and concentrated under reduced pressure to provide the crude product. The crude material was purified by flash chromatography on a silica gel column eluting with 0-10% MeOH/DCM to give 1.2 g desired product. m/z: 777.9 [M+H]⁺.

### Step 6: Synthesis of (S)-2-(3,5-dichloro-4-(8-chloro-5-(2-hydroxy-3-(methylamino)-3-oxopropoxy)-2-methyl-4-oxo-1,6-naphthyridin-1(4H)-yl)phenoxy)ethyl dihydrogen phosphate.

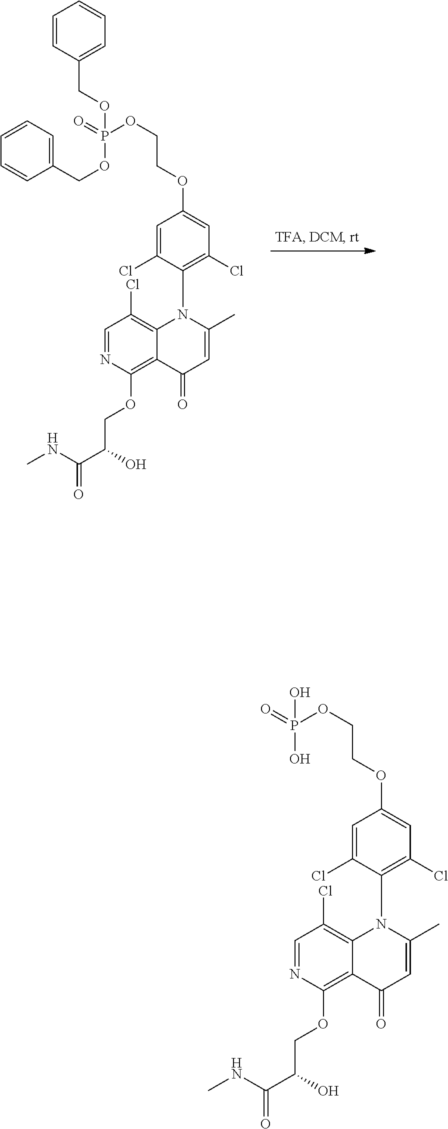

To a solution of (S)-dibenzyl (2-(3,5-dichloro-4-(8-chloro-5-(2-hydroxy-3-(methylamino)-3-oxopropoxy)-2-methyl-4-oxo-1,6-naphthyridin-1(4H)-yl)phenoxy)ethyl) (1.2 g, 1.54 mmol) in DCM (7.0 mL) was added 2.2,2-trifluoroacetic acid (3.0 mL). The reaction mixture was stirred at 23°°C. for 40 hours until completion of the reaction, as shown by LC/MS analysis. The reaction mixture was concentrated under reduced pressure to provide the crude product. The crude material was purified by RP-HPLC (MeCN/water+0.1% TFA at 75 mL/min flow rate, gradient: 10-30% MeCN over 9.5 min, column: Waters XBridge C18 OBD 30×100 mm; 5 micron) to give 0.85 g desired product as an amorphous powder after lyophilization. ¹H NMR (400 MHZ, McOD) δppm 8.21 (s, 1H), 7.29 (s, 2H), 6.54 (s, 1H), 4.63 (qd, J=11.0, 5.1 Hz, 2H), 4.48 (dd, J=5.9, 4.4 Hz, 1H), 4.34 (d, J=3.9 Hz, 4H), 2.82 (s, 3H), 2.04 (s, 3H). m/z: 597.8 [M+H]⁺.

The phosphoric acid moiety of the compound of Example 1 was converted into various salts following the procedures given below in Examples 1a, 1b and 1c.

### Example 1a (Calcium Salt)

(S)-2-(3,5-dichloro-4-(8-chloro-5-(2-hydroxy-3-(methylamino)-3-oxopropoxy)-2-methyl-4-oxo-1,6-naphthyridin-1(4H)-yl)phenoxy)ethyl dihydrogen phosphate (50 mg. 0.084 mmol) was dissolved in 5 mL MeOH and 1 mL water. To the vigorously stirred solution was added 0.3 mL 1.0 M Ca (Oac)₂ dropwise. The suspension was stirred for 10 min after addition. 5 mL MeOH was added and the mixture stayed at room temperature for 2 hours. The precipitate was filtered and washed with MeOH. The product obtained was dried to give 40 mg (S)-2-(3,5-dichloro-4-(8-chloro-5-(2-hydroxy-3-(methylamino)-3-oxopropoxy)-2-methyl-4-oxo-1,6-naphthyridin-1(4H)-yl)phenoxy)ethyl dihydrogen phosphate calcium salt as a white solid. ¹H NMR (400 MHz, DMSO+1 drop TFA) δppm 8.25 (s, 1H), 7.41 (s, 2H), 6.50 (s, 1H), 4.50-4.44 (m, 2H), 4.37-4.26 (m, 3H), 4.20-4.11 (m, 2H), 2.67 (d, J=4.5 Hz, 3H), 1.93 (s, 3H). MS: 597.8 [M+H]⁺.
Anal. Calcd. For C₂₁H₁₉CaCl₃N₃O₉P: C, 39.73; H, 3.02; Ca, 6.31; Cl 16.75; N, 6.62; O, 22.68; P, 4.88. Found Caled. For C₂₁H₁₉CaCl₃N₃O₉P: Calcium ion analysis (ICP-OES): 4.76.

### Example 1b (Sodium Salt)

(S)-2-(3,5-dichloro-4-(8-chloro-5-(2-hydroxy-3-(methylamino)-3-oxopropoxy)-2-methyl-4-oxo-1,6-naphthyridin-1(4H)-yl)phenoxy)ethyl dihydrogen phosphate (800 mg, 1.34 mmol) was dissolved in 3 mL MeOH and 7 mL water. To the stirred solution was added 3 mL 1.0 M NaOAc dropwise. The obtained solution was purified by RP-flash chromatography on a C18 column with MeCN/water gradient from 2 to 90% two times to give (S)-2-(3,5-dichloro-4-(8-chloro-5-(2-hydroxy-3-(methylamino)-3-oxopropoxy)-2-methyl-4-oxo-1,6-naphthyridin-1(4H)-yl)phenoxy)ethyl dihydrogen phosphate mono sodium salt. The fractions were pooled and lyophilized. The amorphous powder obtained was dissolved in MeOH and concentrated to dryness. The residue was treated with 20 mL acetonitrile. The resulting suspension was sonicated for 20 min and remained at room temperature for 10 min. It was filtered and dried in a vacuum oven at 80° C. for 40 hours to give 300 mg desired product as fine white powder. ¹H NMR (400 MHZ, McOD) δppm 8.21 (s, 1H), 7.27 (s, 2H), 6.53 (s, 1H), 4.69-4.57 (m, 2H), 4.48 (t, J=5.1 Hz, 1H), 4.30 (t, J=4.8 Hz, 2H), 4.26-4.16 (m, 2H), 2.82 (s, 3H), 2.04 (s, 3H). MS: 597 8 [M+H]⁺.
Anal. Calcd. For C₂₁H₂₀Cl₃N₃NaO₉P: C. 40.77; H, 3.26; Cl, 17 19; N, 6.79; Na, 3.72; O, 23.27; P, 5.01. Found Calcd. For C₂₁H₂₀Cl₃N₃NaO₉P: Sodium ion analysis (ion chromatography): 5.03.

### Example 1c (Sodium Salt)

To a solution of (S)-2-(3,5-dichloro-4-(8-chloro-5-(2-hydroxy-3-(methylamino)-3-ovopropoxy)-2-methyl-4-oxo-1,6-naphthyridin-1 (4H)-yl) phenoxy) ethyl dihydrogen phosphate mono sodium salt (200 mg, 0.32mmol) in 5 mL water was added sodium hydroxide (1.0 M, 0.323 mL). The obtained solution was purified by RP-flash chromatography on a C18 column with MeCN/water gradient from 5 to 100% to provide 2 major peaks. The fractions of peak 1 (pH of solution ˜7) were pooled and lyophilized to dryness. NMR analysis indicated around 90% desired product and 10% undesired regioisomer ((S)-2-(3,5-dichloro-4-(8-chloro-5-((3-hydroxy-1-(methylamino)-1-oxopropan-2-yl) oxy)-2-methyl-4-oxo-1,6-naphthyridin-1(4H)-yl)phenoxy)ethyl dihydrogen phosphate). The material was repurified by RP-flash chromatography on a C18 column with a MeCN/water gradient from 5 to 100% to give 2 major peaks again. The fractions of peak 1 were pooled and lyophilized to dryness. NMR analysis indicated pure product(S)-2-(3,5-dichloro-4-(8-chloro-5-(2-hydroxy-3-(methylamino)-3-(oxopropoxy)-2-methyl-4-oxo-1,6-naphthyridin-1(4H)-yl)phenoxy)ethyl dihydrogen phosphate di-sodium salt. The amorphous powder obtained was dissolved in MeOH and concentrated to dryness. The residue was treated with 20 mL acetonitrile. The resulting suspension was sonicated for 20 minutes and remained at room temperature for 10 minutes. It was filtered and dried in a vacuum oven at 90° C. for 16 hours to give 70 mg desired product as fine white powder. ¹H NMR (400 MHZ, MeOD) δppm 8.21 (s, 1H), 7.27 (s, 2H), 6.54 (s, 1H), 4.69-4.59 (m, 2H), 4.49 (t, J=5.1 Hz, 1H), 4.30 (t, J=5.0 Hz. 2H), 4.19 (dt, J=7.1, 5.2 Hz, 2H), 2.82 (s. 3H), 2.04 (s, 3H). MS: 597.8 [M+H]⁺.
Anal. Calcd. For C₂₁H₁₉Cl₃N₃Na₂O₉P: C, 39.37; H, 2.99; Cl, 16.60; N, 6.56; Na, 7.18; O, 22.47; P, 4.83. Found Calcd. For C₂₁H₁₉Cl₃N₃Na₂O₉P: Sodium ion analysis (ion chromatography): 7.86.

### Example 2: Synthesis of (S)-3-((8-chloro-1-(2,6-dichloro-4-(2-hydroxyethoxy)phenyl)-2-methyl-4-oxo-1,4-dihydro-1,6-naphthyridin-5-yl) oxy)-1-(methylamino)-1-oxopropan-2-yl dihydrogen phosphate.

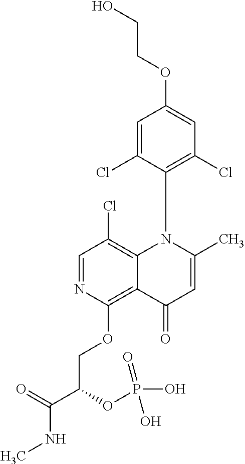

### Step 1: Synthesis of (S)-2-(4-(5-((3-((tert-butyldiphenylsilyl)oxy)-1-(methylamino)-1-oxopropan-2-yl)oxy)-8-chloro-2-methyl-4-oxo-1,6-naphtbyridin-1(4H)-yl)-3,5-dichlorophenoxy)ethyl acetate

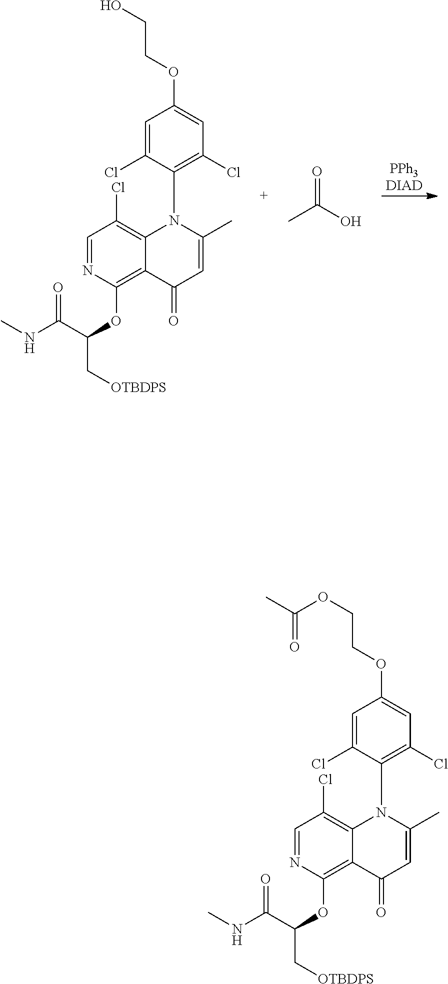

To a solution of(S)-3-((tert-butyldiphenylsilyl) oxy)-2-((8-chloro-1-(2,6-dichloro-4-(2-hydroxyethoxy)phenyl)-2-methyl-4-oxo-1,4-dihydro-1,6-naphthyridin-5-yl)oxy)-N-methylpropanamide (1000 mg, 1.32 mmol) in DCM (15.0 mL) was added triphenylphosphine (382 mg, 1.46 mmol), acetic acid (95 mg, 1.59 mmol) and DIAD (294 mg. 1.46 mmol). The mixture was stirred at room temperature for 16 hours. LC/MS showed completion of the reaction. The mixture was purified by flash column using 0-100% EtOAc/heptane as an eluent to give 780 mg product. m/z: 796.2/798.2 [M+H]⁺.

### Step 2: Synthesis of(S)-2-(3,5-dichloro-4-(8-chloro-5-(2-hydroxy-3-(methylamino)-3-oxopropoxy)-2-methyl-4-oxo-1,6-naphthyridin-1 (4H)-yl) phenoxy) ethyl acetate

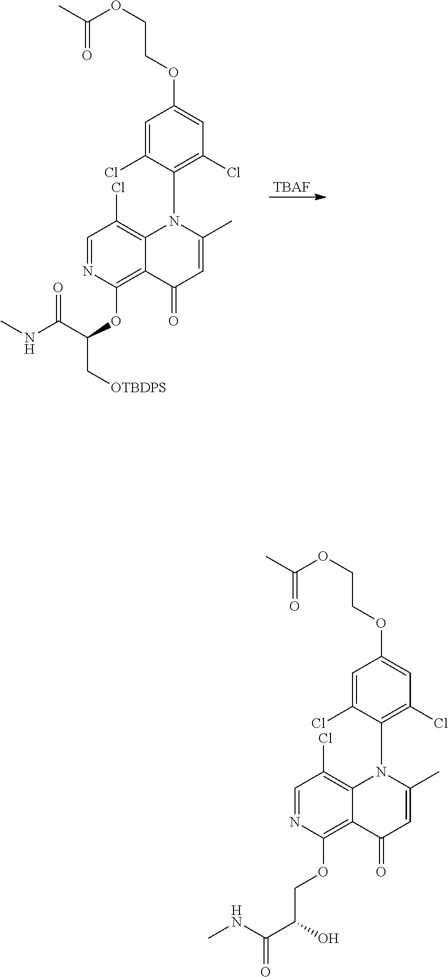

To a solution of (S)-2-(4-(5-((3-((tert-butyldiphenylsilyl)oxy)-1-(methylamino)-1-oxopropan-2-yl)oxy)-8-chloro-2-methyl-4-oxo-1,6-naphtbyridin-1(4H)-yl)-3,5-dichlorophenoxy) ethyl acetate (780 mg, 0.98 mmol) in 10 mL THF was added tetrabutylammonium fluoride (2.94 mL, 1.0 M, 2.94 mmol) at 0° C. dropwise quickly. The ice bath was removed after addition, and the reaction mixture was stirred at 23° C. for 2 hours. LC/MS showed completion of the reaction. The reaction mixture was diluted with EtOAc and washed with water. The layers were separated and the aqueous layer was extracted with EtOAc twice. The organic layers were combined, washed with brine and dried over anhydrous MgSO₄, concentrated under reduced pressure to provide 546 mg of crude product which was used in the next step without purification. m/z: 558.0/560.0 [M+H]⁺.

### Step 3: Synthesis of(S)-2-(4-(5-(2-((bis(benzyloxy)phosphoryl)oxy)-3-(methylamino)-3-oxopropoxy)-8-chloro-2-methyl-4-oxo-1,6-naphthyridin-1(4H)-yl)-3,5-dichlorophenoxy)ethyl acetate

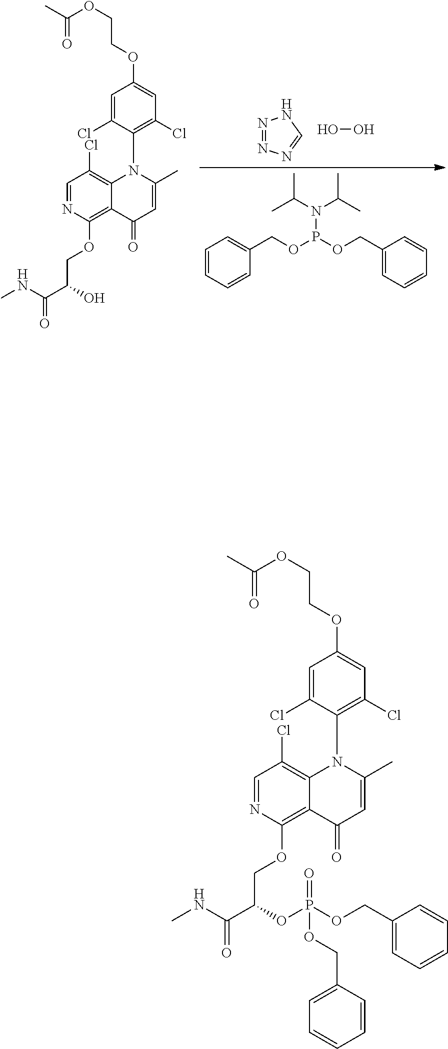

To a solution of (S)-2-(3,5-dichloro-4-(8-chloro-5-(2-hydroxy-3-(methylamino)-3-oxopropoxy)-2-methyl-4-oxo-1,6-naphthyridin-1(4H)-yl)phenoxy)ethyl acetate (546.0 mg, 0.98 mmol) in DCM (2.0 mL) and 1H-tetrazole (3.7 mL, 0.4 molar, 1.48 mmol) at 0° C. was slowly added dibenzyl diisopropylphosphoramidite (405 mg, 1.17 mmol). The mixture was allowed to warm to room temperature and stirred for 2 hours. The mixture was cooled to 0° C. and quenched with hydrogen peroxide (0.3 mL, 30% wt, 2.9 mmol). It was allowed to warm to room temperature and stirred for 15 mins. LC/MS showed completion of the reaction. The mixture was concentrated under reduced pressure and the residue was diluted with EtOAc, washed with water and brine, dried over anhydrous MgSO₄, and concentrated under reduced pressure to give crude product. The crude material was purified by flash colum using 0-10% MeOH/DCM as an eluent to give 420 mg product (around 80% pure based on UV analysis at 214 nm). m/z: 818.0/820.0 [M+H]⁺.

### Step 4: Synthesis of (S)-dibenzyl (3-((8-chloro-1-(2,6-dichloro-4-(2-hydroxyethoxy) phenyl)-2-methyl-4-oxo-1,4-dihydro-1,6-naphthyridin-5-yl)oxy)-1-(methylamino)-1-oxopropan-2-yl)phosphate

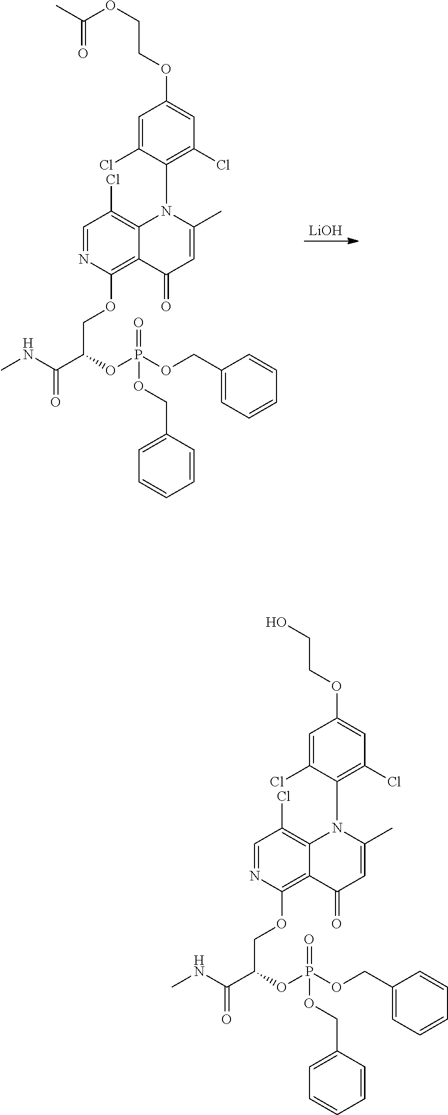 [SMILES: CNC(=O)[C@H](COc1ncc(Cl)c2c1c(=O)cc(C)n2-c1c(Cl)cc(OCCOC(C)=O)cc1Cl)OP(=O)(OCc1ccccc1)OCc1ccccc1 ; "LiOH" ; CNC(=O)[C@H](COc1ncc(Cl)c2c1c(=O)cc(C)n2-c1c(Cl)cc(OCCO)cc1Cl)OP(=O)(OCc1ccccc1)OCc1ccccc1]

To a solution of (S)-2-(4-(5-(2-((bis(benzyloxy)phosphoryl)oxy)-3-(methylamino)-3-oxopropoxy)-8-chloro-2-methyl-4-oxo-1,6-naphthyridin-1(4H)-yl)-3.5-dichlorophenoxy)ethyl acetate (420.0 mg, 80% wt, 0.41 mmol) in MeOH (5.000 mL) was added lithium hydroxide (1.0 mL, 1.0 M, 1.0 mmol) and the mixture was stirred at room temperature for 16 hours. LC/MS showed completion of the reaction. 1.0 mL 1.0 M HCl was added, filtered and the filtrate was purified by HPLC (C18 30×50 mm, 5 μm; solvent A: water+0.1% formic acid, solvent B: ACN+0.1% formic acid; B/A 35-60%) to give 140 mg product. ¹H NMR (400 MHz. MeOD) δppm 8.14 (s, 1H), 7.33 (d, J=3.0 Hz, 5H), 7.26 (s, 7H), 6.46 (s, 1H), 5.13-5.09 (m, 3H), 5.06 (dd, J=8.5, 2.2 Hz, 2H), 4.80 (dd, J=12.0, 3.8 Hz, 1H), 4.65 (dd, J=12.1, 4.5 Hz, 1H), 4.19-4.15 (m, 2H), 3.93-3.88 (m, 2H), 2.78 (s, 3H), 2.01 (s, 3H). m/z: 776.2/778.2 [M+H]⁺.

### Step 5: Synthesis of (S)-3-((8-chloro-1-(2,6-dichloro-4-(2-hydroxyethoxy)phenyl)-2-methyl-4-oxo-1,4-dihydro-1,6-naphthyridin-5-yl)oxy)-1-(methylamino)-1-oxopropan-2-yl dihydrogen phosphate.

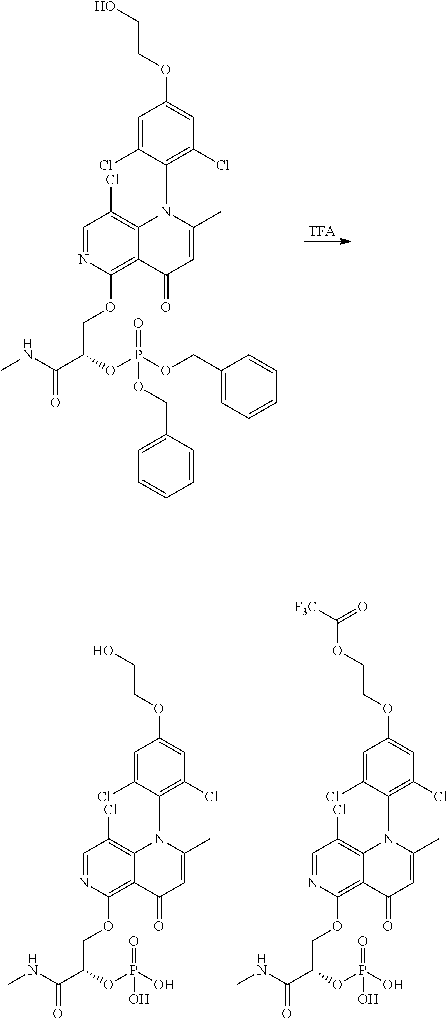

To a solution of (S)-dibenzyl (3-((8-chloro-1-(2,6-dichloro-4-(2-hydroxyethoxy)phenyl)-2-methyl-4-oxo-1,4-dihydro-1,6-naphthyridin-5-yl)oxy)-1-(methylamino)-1-oxopropan-2-yl) phosphate (140.0 mg. 0.18 mmol) in DCM (1.4 mL) was added TFA (600 μL, 7.75 mmol). The mixture was stirred at room temperature for 16 hours and concentrated under reduced pressure to give crude product. The crude mixture was purified by HPLC (C18 30×50 mm, 5 μm; solvent A: water+0.1% formic acid; solvent B: ACN+0.1% formic acid; B/A 10-90%) to give 12 mg desired product (S)-3-((8-chloro-1-(2,6-dichloro-4-(2-hydroxyethoxy)phenyl)-2-methyl-4-oxo-1,4-dihydro-1,6-naphthyridin-5-yl)oxy)-1-(methylamino)-1-oxopropan-2-yl dihydrogen phosphate. ¹H NMR (400 MHZ, MeOD) δppm 8.22 (s, 1H), 7.27 (s, 2H), 6.56 (s, 1H), 5.10 (ddd, J=9.7, 7.3, 2.8 Hz, 1H), 5.00 (dd, J=11.7, 2.7 Hz, 1H), 4.54 (dd, J=11.7, 7.3 Hz, 1H), 4.20-4.14 (m, 2H), 3.94-3.88 (m, 2H), 2.83 (s, 3H), 2.04 (s, 3H). m/z: 596.0/598.0 [M+H]⁺. 95 mg side product (S)-2-(3,5-dichloro-4-(8-chloro-2-methyl-5-(3-(methylamino)-3-oxo-2-(phosphonooxy)propoxy)-4-oxo-1,6-naphthyridin-1 (4H)-yl)phenoxy)ethyl 2,2,2-trifluoroacetate was also obtained. m/z: 692.2/694.2 [M+H]⁺.

### Example 2a (Sodium Salt)

(S)-2-(3,5-dichloro-4-(8-chloro-2-methyl-5-(3-(methylamino)-3-oxo-2-(phosphonooxy0propoxy)-4-oxo-1,6-naphthyridin-1(4H)-yl)phenoxy)ethyl 2,2,2-trifluoroacetate was converted into the sodium salt of the compound of Example 2 following the procedure given below.

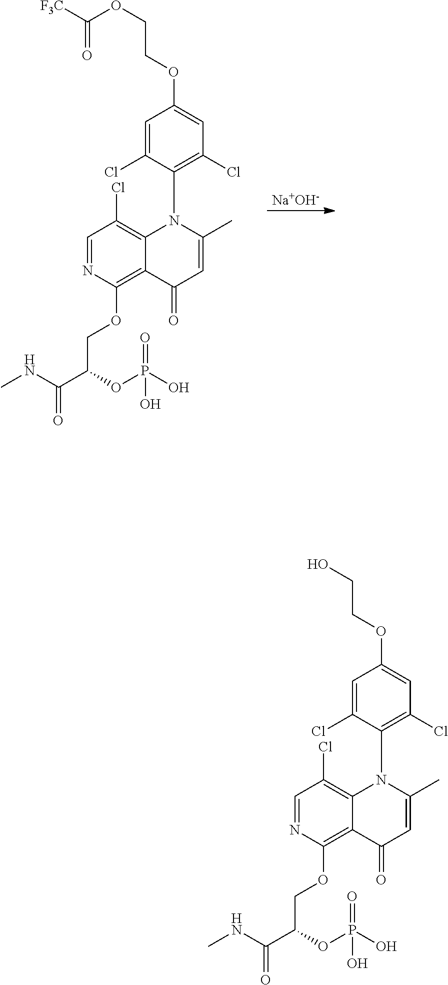

To a suspension of(S)-2-(3,5-dichloro-4-(8-chloro-2-methyl-5-(3-(methylamino)-3-oxo-2-(phosphonooxy)propoxy)-4-oxo-1,6-naphthyridin-1(4H)-yl)phenoxy)ethyl 2,2,2-trifluoroacetate (95 mg, 137 μmol) in water (2.0 mL) was added NaOH (0.55 mL, 1.0 M, 550 μmol) and the mixture was stirred at room temperature for 2 hours. The mixture was purified by reverse phase flash chromatography with 10-30% acetonitrile/water as eluent. The fractions which contained product were pooled and subsequent freeze drying provided 68 mg product as sodium salt of the compound of Example 2. ¹H NMR (400 MHZ, MeOD) δppm 8.11 (s, 1H), 7.31-7.21 (m, 2H), 6.54 (s, 1H), 4.96-4.89 (m, 2H), 4.73-4.62 (m, 1H), 4.21-4.14 (m, 2H), 3.91 (dd, J=5.2, 4.0 Hz, 2H), 2.77 (s, 3H), 2.01 (s, 3H). m/z: 596.1/598.1 [M+H]⁺.

### Example 3: In Vitro Conversion of the Pro-Drug Compounds of the Present Disclosure to the Parent Compounds by Intestinal S9 Fraction Across Species

### Assay Protocol

Drug stock solutions (100 μM) were prepared in methanol by two subsequent 1:10 dilutions from 10 mM DMSO drug solutions.

Frozen pooled intestinal S9 fractions (final protein concentration 2 mg/mL), alamethicin (25 μg/mg). 0.IM phosphate buffer containing 1 mM magnesium chloride and NADPH (final concentration 1 mM) plus UDPGA (final concentration 3.5 mM) are pre incubated at 37° C. prior to the addition of test compound (final substrate concentration 1 μM; final DMSO concentration 0.25%) to initiate the reaction. An additional incubation is performed in the absence of cofactors (NADPH, UDPGA).

Incubation plate was incubated on a shaker with temperature controlled at 37° C. Sequential samples were removed at designated timepoints (0, 5, 15, 30, 60 and 120 mins) and quenched with 4 volumes of ice-cold acetonitrile (containing internal standard glyburide). At the end of the incubation, samples plates were centrifuged, and supernatants reconstituted in water. Samples were then analyzed by LC/MS to assess parent depletion.

### Assay Conditions

| Compound concentration | Nominally 1 μM | |
|---|---|---|
| Intestinal S9 concentration | 2 | mg/mL |
| Strain/Species | CD-1 Mouse, Sprague | |
| | Dawley Rat, Beagle Dog, | |
| | Cynomolgous Monkey and Human | |
| Gender | Mixed gender for human, all | |
| | other species were male | |
| NADPH concentration | 1 | mM |
| UDGPA concentration | 3.5 | mM |

| | |
|---|---|
| Time-points (n = 1) | 0, 5, 15, 30, 60, 120 minutes |
| Incubation conditions | 37° C. atmospheric air, 300 rpm |

### Data Analysis

A ratio of compound peak area/internal standard peak area for each time point is generated and subsequently expressed as % of the response at time 0. The percentage stability of a compound in intestinal S9 solution at time t is calculated as detailed in Equation 1:

$$\\ \begin{matrix}\% Stability = \frac{PARt}{PARt = 0} \times 100 & Equation 1\end{matrix}$$

The stability data is transformed using a non-linear regression (XLfit model “Exp/Log 500”) and plotted against time points to define the initial phase of the slope (or initial rate of elimination kel).

The half-life (t_{1/2}) is calculated using the Equation 2:

$$\\ \begin{matrix}Half ‐ life ( min ) = \frac{LN ( 2 )}{kel} & Equation 2\end{matrix}$$

The intrinsic clearance (Clint) is calculated using the Equation 3:

$$\\ \begin{matrix}Clint ( uL / min / mg ) = \frac{kel}{Cprotein} & Equation 3\end{matrix}$$

### Results

T_{1/2} of conversion of the compound of Example 1a to the parent compound in rat intestinal S9=14.4 min (+ cofactor), 16.5 min (− cofactor); in dog intestinal S9=45.3 min (+ cofactor), 46 min (− cofactor); in human intestinal S9=88.7 min (+ cofactor), 86.7 min (− cofactor).

### Example 4: GIRK1/4 In Vitro Activity Assay

The GIRK1/4 activity of the parent compounds formed after cleavage upon hydrolysis of the phosphoric acid ester group of the compounds according to the present disclosure was assessed by the following in vitro method.

### Buffers

a. External buffer: 10 mM NaCl, 50 mM Na Gluconate, 80 mM K Gluconate, 1.8 mM CaCl₂, 1 mM MgCl₂, 10 mM HEPES, 10 mM Glucose, pH 7.4; osmolarity 300-310 Osm/L.
b. Internal buffer: 30 mM KCl, 100 mM K Gluconate, 1 mM MgCl₂, 10 mM HEPES, 1 mM EGTA, 10 mM NaCl, pH 7.2; osmolarity 284-292 Osm/L.

### Compounds

c. Prepare seven-fold compound dilution series (10 mM to 20 μM) in 100% DMSO, in 384-well polypropylene plates.
d. Propafenone (Sigma Aldrich, catalog number P4670) was used as a positive control and DMSO for neutral control.
e. Resuspend 1 μl of compounds in DMSO into 66.7 μl external buffer in 384-well polypropylene plate and load into Plate 1 section of Molecular Devices Quattro.

### Quattro Setup

f. Load 384-well Population Patch Plate (Molecular Devices #9000-0902) into Quattro.
g. Fill Quattro F-soak trough with 20% DMSO and 50% EtOH.
h. Fill Quattro buffer trough with external buffer.
i. Attach internal buffer flask to Quattro internal buffer tubing.
j. Attach PBS (Phosphate Buffered Saline, minus Ca⁺⁺ and Mg⁺⁺, pH7.4) bottle to F-head & E-head wash on Quattro.

### Antibiotic

k. Resuspend 5.1-5.8 mg amphotericin B (Sigma Aldrich, catalog number A2411) in 175 μl DMSO.
l. Add the resulting solution to 50 mL internal buffer and attach to antibiotic tubing port on Quattro.

### Cells

m. Used GIRK 1/4 HEK293 stable cells (obtained from ChanTest, 14656 Neo Parkway, Cleveland, Ohio 44128) grown to ˜80% confluency in the following cell culture medium: DMEM containing 10% (v/v) Fetal Bovine Serum, Penicillin/Streptomycin (at “1×” concentration from a 100× stock), 0.5 mg/mL G418 and 0.1 mg/mL Zeocin.
n. Detached cells using Detachin (Genlantis, 11011 Torreyana, San Diego, CA 92121), washed with PBS (minus Ca⁺⁺ and Mg⁺⁺) and resuspended in extemal buffer (5 mL final volume at 2.0-2.1×10⁶ cells/mL.).
o. Loaded into cell trough of Quattro.

### Assay Protocol

Quattro was controlled using IonWorks v2 software to perform the following steps:
p. Added 3.5 μl cells plus 3.5 μl external buffer to wells of Quattro Patch Plate.
q. Circulated amphotericin B and internal buffer onto cells.
r. Applied the following voltage protocol: Pulse 1: 15 mV for 300 milliseconds (ms), followed by Pulse 2: −120 mV for 400 ms. then Pulse 3:-15 mV for 400 ms, and finally Pulse 4: −120 mV to 40 mV over 500 ms (this is a voltage ramp).
s. Measured magnitude of inward potassium current at time point between 1200-1220 ms from start of Pulse 1 (i.e. during the voltage ramp phase).
t. Added 3.5 μl of diluted compounds (or DMSO) to wells and repeated steps c-d (final compound concentrations are 50 uM to 0.1 uM, and each concentration was tested in quadruplicate—i.e. in 4 separate wells).
u. The difference between current magnitude pre vs. post-compound gave a measure of GIRK1/4 inhibition.

### Data Analysis

IC₅₀ values were calculated by plotting the percentage of current inhibition (normalized to the DMSO-only control) as a function of compound concentration using standard data analysis software.

### Results

Using the test assay as described above the parent compound of the compounds of Examples 1 and 2 of the disclosure exhibited inhibitory activity (IC₅₀) of 0.06 μM. The compounds of Examples 1 and 2 themselves have IC₅₀ of >10 μM.

### Example 5: In Vivo Pharmacokinetic (PK)/Pharmacodynamic (PD) Behaviour of the Parent Compounds in the Guinea Pig Model

The pharmacodynamic behaviour of parent compounds formed after cleavage upon hydrolysis of the phosphoric acid ester group of the compounds according to the present disclosure was assessed by the following in vivo method.

1) Guinea pigs Studies described in this report were performed according to approved Novartis Institutional Animal Care and Use Committee (IACUC) protocols and were in compliance with the Animal Welfare Act Regulations 9 CFR Parts 1, 2, and 3, and other guidelines [Guide for the Care and Use of Laboratory Animals, 1995] Female Hartley guinea pigs (Hartley) of 550-650 grams were purchased from Charles River. Animals in this body weight range were chosen as they have optimal parameters for the assay, including size of trachea, blood vessels, and ideal cardiovascular health. Upon arrival guinea pigs were housed five per cage in a specific pathogen-free facility with a 12-hour regular light cycle (6:00-18:00) and with free access to normal chow diet and water ad libitum. Animals were acclimated for at least 7 days before being used in the experiment. They were housed on a 12-hour light/dark cycle and provided guinea pig normal research diet and water ad libitum.

Guinea pigs were anesthetized with an intraperitoneal injection of Ketamine/Xylazine (85/15 mg/kg) and maintained with inhalation of isoflurane 2-3% (in oxygen) to effect via nose cone. The level of surgical anesthesia was assessed by monitoring of the pedal pinch reflex. Under anesthesia, animals were placed on a heating pad (non-electric) controlled to 37-39° C. A temperature probe was inserted into rectum for constant body temperature monitoring. Animals were prepared by shaving the neck and extremities, and placing a pulse-oximetry sensor (typically on the hind foot) for monitoring blood oxygen level. Surface electrocardiogram (ECG) Lead-II was recorded by connecting 3 pin-probes into the left hind limb and the right/left forelimbs subcutaneously and was used for monitoring HR and presence of atrial or ventricular arrhythmias. A peripheral vein catheterization was conducted to enable intravenous (iv) drug administration. A rubber band was tied upstream of the insertion site (typically the cephalic vein) and a 24G×¾″ iv catheter with 27G needle inserted into the vein. Once blood entered the catheter, the 27G needle was gently pulled out then advanced. The rubber band was released and sterile beparin saline solution (10 U/mL, 0.1 mL) injected to test catheter patency. A sterile adaptor with a Luer lock pre-filled with heparin saline (10 U/mL) was attached to the catheter end, and the catheter was fixed to the limb using tape.

2) Surgical Procedures. With animals in a deep surgical plane of anesthesia. endotracheal intubation was established by making a longitudinal incision (˜1-1.5 cm in length) through the skin on the ventral midline aspect of the neck. Blunt dissection was then used to expose and isolate the trachea (while avoiding disruption of the recurrent laryngeal nerve). A transverse tracheal incision (˜3 mm) was made on the ventral aspect of the trachea between two cartilage rings below the level of the larynx. An appropriately sized endotracheal tube (OD 2.9 mm/ID 2.0 mm) was inserted into the trachea and advanced ˜1-1.5 cm (not beyond the level of tracheal bifurcation). Once in place, the tube was secured via placement of a 4-0 silk encircling suture. The endotracheal tube was connected to a ventilator (Harvard Apparatus, Inc. model #683) and a pulmonary pressure monitor/limiter (Harvard Apparatus, Inc. Type 870/1). The nose cone delivering isoflurane was disconnected at this point. and isoflurane (in oxygen) was instead delivered to effect through the ventilator from this point and for the remainder of the procedures. Ventilation was set to deliver a tidal volume equivalent to 5-7 mL/kg at ˜55-65 respirations/per minute (requiring the oxygen regulator to be adjusted to ˜0.8 L/min) and to maintain inhalation pressure less than 22 mmHg.

The left carotid artery was cannulated for access to arterial circulation for blood sampling and blood pressure (BP) measurements as follows. The carotid artery was located in the space between the muscle around the trachea and the stern-mastoid muscle. Using fine forceps the vessel was carefully separated from surrounding tissue by blunt dissection to expose a 1-2 cm length of artery. Two sutures (3-0) or 4-0 silk) were placed underneath the vessel, one at the cranial. and the other at the caudal end. The cranial suture was tied around the vessel and weighed down with hemostats. An additional set of hemostats were used to pull the vessel straight from the caudal end (this also ensured that blood flow was temporarily blocked so it did not flow out upon incision). Approximately 13 cm of sterile catheter (micro- urethane tubing OD 0.04/ID 0.025 inches) was used filled with sterile heparin saline solution (10 U/mL) via a sterile lec syringe with 22 gauge Luer stub adapter The vessel was lifted, a small 45° angle incision made, and the catheter inserted to the 2.5-3 cm mark and secured with a caudal suture. Blood flow was checked by withdrawing blood into the catheter then the catheter was attached to a BP sensor (ADInstruments, Inc. Code #MLT844) via an adaptor.

An octapolar electrode recording/pacing probe was used to measure the atrial effective refractory period (AERP). This was accomplished by making an incision (˜1-1.5 cm) on the right ventral aspect of the neck. Using blunt dissection, the right jugular vein was identified, isolated and secured using cranial and caudal ligatures (#4-0 silk). After the cranial suture was tied around the vessel. a venotomy was made between the ligatures. A 1.9F octapolar electrophysiological probe (Transonic Science Inc. Model #FTS-1913A-1018) was inserted and advanced through the vein to achieve optimal placement (i.e., into the right atrium. but not as far as the right ventricle). Upon proper placement as judged from detection of a robust atrial electrogram signal concurrent with the P wave of the surface ECG (indicating atrial depolarization), the probe was secured in place using the caudal ligature.

3) Electrophysiological measurements. AERP was measured using programmed electrical stimulation in a multi-step process. In the first step, the atrium was artificially paced by using an electrode to introduce an electrical stimulus (called S1) at regular intervals. Each S1 stimulus leads to depolarization of the atrial myocardium, which was detected in an atrial electrogram recorded by the same electrode. In the next step, after a series of regular S1 pulses, a premature stimulus (called S2) was introduced, and the atrial electrogram consulted to determine whether atrial depolarization occurred (so-called “capture” of the S2 stimulus). In the final step, the interval between S1 and S2 was increasingly shortened, until S2 no longer resulted in capture. This time period in milliseconds was considered the AERP duration.

In the guinea pig model, right AERP was measured multiple times in the same animal over the course of the experiment, by recording an atrial electrogram while simultaneously electrically pacing the right atrium using the octopolar probe. Specifically, after ˜10-20 minutes of stabilization after implanting the electrode, the first AERP was measured using a PC-based four-channel programmable pulse generator (ADInstruments, Inc.). Each S1 pulse was rectangular in shape, lasted for 2 milliseconds and was delivered at 0.5 to 3 volts (as needed to cause atrial depolarization) and a basal cycle length between 150 to 300 milliseconds (as needed to capture successive S1 stimuli reliably). The premature stimulus (S2) was introduced after a cycle of 10 recurring S1 stimuli and capture evaluated on the atrial electrogram. The time between the last S1 and S2 was reduced by 2 msec for each subsequent cycle until S2 excitation failed to depolarize the atria. The AERP was defined as the longest S1-S2 interval where the S2 failed to capture.

AERP was measured multiple times in the same animal as follows: (1) at baseline prior to any compound administration (3 to 4 AERP measurements): (2) at basal state after dosing fingolimod (intravenous, 0.1 mg/kg, 0.25 mL/kg) to open GIRK1/4 channels and shorten AERP (3 to 4 AERP measurements, 5 minutes apart) and (3) after intravenous infusion of test compound, (at 5, 10, 15, 20, 30, 40, 50 and 60 minutes after starting the infusion).

Test compound was formulated in N-methyl-2-pyrrolidone (NMP, 10% v/v), polyethylene glycol 300 (PEG300, 10% v/v), 20% Solutol HS15 (50% v/v) and 5% Pluronic F-68 (30% v/v). Intravenous infusion started at ˜5 minutes post-formulation and proceeded at a rate of 50 μL/min/kg for 60 minutes using an infusion pump through a surface venous catheter (Section 2.4.2). Test compound was diluted in vehicle to achieve doses of 0.09, 0.3, 0.9, 3 and 9 mg/kg.

Blood samples (˜60 μl) were collected from the carotid artery catheter following AERP measurements at 5, 10, 15, 20, 30, 40, 50 and 60 minutes after starting infusion of test compound to establish a PK/PD relationship. The carotid artery catheter was flushed with 0.1 mL of heparin saline solution (10 U/mL.) between collections to ensure patency. Blood samples were collected into EDTA coated tubes, placed on ice and centrifuged at 13,000 rpm for 10 min at 4°° C. in an Eppendorf centrifuge. Plasma samples were transferred into a 96-deep well plate and maintained at −80° C. for PK analysis.

4) Data Analysis. AERP, as measured in milliseconds, was normalized to baseline and basal (i.e., after opening GIRK1/4 channels with fingolimod) values in order to enable comparisons between doses and animals. AERP duration at any given time after administration of the test compounds was normalized to “% restoration” with the following formula: [(post-test compound value−basal value)/(baseline value−basal value)]×100. Percent restoration values and corresponding total plasma concentrations were then used to calculate EC₅₀ and EC₉₀ values. EC₅₀ and EC₉₀ values were calculated using the GraphPad Prism 7.03 non-linear fit log (inhibitor) vs. response-variable slope (four-parameter) analysis.

### Equivalents

Those skilled in the art will recognize, or be able to ascertain, using no more than routine experimentation, numerous equivalents to the specific embodiments described specifically herein. Such equivalents are intended to be encompassed in the scope of the following claims.

1. A compound which is a phosphoric acid ester derivative of a compound of formula (I): 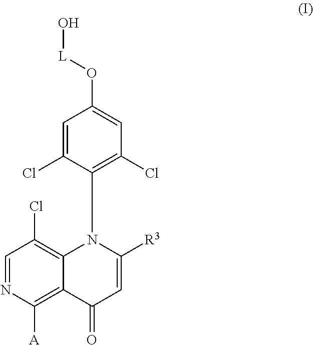
or a pharmaceutically acceptable salt thereof, wherein
L is (C₁-C₆) alkylene optionally substituted with one or more —OH;
A is —OR² or (C₁-C₆) alkyl optionally substituted with one or more substituents independently selected from —SO₂(C₁-C₄)alkyl, —NHC(O)R^{b}, and —C(O)NHR^{c};
R² is (C₁-C₆) alkyl substituted with one or more substituents independently selected from —NHC(O)R^{d} and —C(O)NHR^{e}, wherein the (C₁-C₆)alkyl is optionally further substituted with one or more substituents independently selected from halogen, —OH and —CN;
R^{a}, R^{b}, R^{c}, R^{d}, and R^{e} are each independently selected from H and (C₁-C₆) alkyl optionally substituted with one or more —OH; and
R³ is (C₁-C₄)alkyl.

2. A compound according to claim 1 or a pharmaceutically acceptable salt thereof, wherein L is selected from —CH₂CH₂—, —CH₂—CH(CH₃)—, and —CH₂C(CH₃)₂—.

3. A compound according to claim 1 or a pharmaceutically acceptable salt thereof, said compound having formula (II): 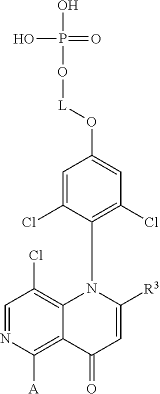
wherein
L is (C₁-C₆) alkylene optionally substituted with one or more —OH;
A is —OR² or (C₁-C₆)alkyl optionally substituted with one or more substituents independently selected from —SO₂(C₁-C₄)alkyl, —NHC(O)R^{a}, and —C(O)NHR^{b};
R² is (C₁-C₆) alkyl substituted with one or more substituents independently selected from —NHC(O)R^{c} and —C(O)NHR^{d}, wherein the (C₁-C₆) alkyl is optionally further substituted with one or more substituents independently selected from halogen, −OH and −CN;
R^{a}, R^{b}, R^{c} and R^{d} are each independently selected from H and (C₁-C₆) alkyl optionally substituted with one or more —OH; and
R³ is (C₁-C₄) alkyl.

4. A compound according to to claim 1 or a pharmaceutically acceptable salt thereof, wherein A is —OR².

5. A compound according to claim 1 or a pharmaceutically acceptable salt thereof, said compound having formula (IV): 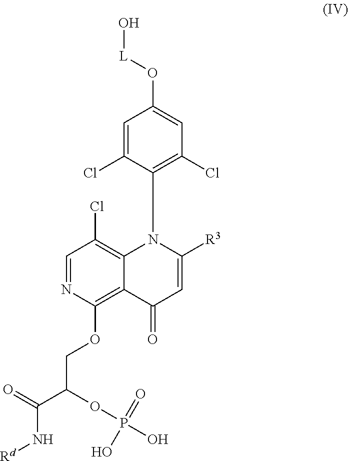
wherein
L is (C₁-C₆)alkylene optionally substituted with one or more —OH;
R^{d} is selected from H and (C₁-C₆) alkyl optionally substituted with one or more —OH; and
R³ is (C₁-C₄)alkyl.

6. A compound according to claim 1 or a pharmaceutically acceptable salt thereof, wherein R³ is —CH₃.

7. A compound according to claim 1 or a pharmaceutically acceptable salt thereof, said compound being a phosphoric acid ester derivative of a compound of formula (1) selected from the group consisting of:
3-((8-chloro-1-(2,6-dichloro-4-(2-hydroxyethoxy)phenyl)-2-methyl-4-oxo-1,4-dihydro-1,6-naphthyridin-5-yl) oxy)-N-methylpropanamide;
2-((8-chloro-1-(2,6-dichloro-4-(2-hydroxyethoxy)phenyl)-2-methyl-4-oxo-1,4-dihydro-1,6-naphthyridin-5-yl) oxy)-N-(2-hydroxyethyl) acetamide;
3-((8-chloro-1-(2,6-dichloro-4-(2-hydroxy-2-methylpropoxy)phenyl)-2-methyl-4-oxo-1,4-dihydro-1,6-naphthyridin-5-yl) oxy)-N-methylpropanamide;
2-((8-chloro-1-(2,6-dichloro-4-(2-hydroxy-2-methylpropoxy)phenyl)-2-methyl-4-oxo-1,4-dihydro-1,6-naphthyridin-5-yl) oxy)-N-methylacetamide;
2-((8-chloro-1-(2,6-dichloro-4-(2-hydroxyethoxy)phenyl)-2-methyl-4-oxo-1,4-dihydro-1,6-naphthyridin-5-yl) oxy)-N-methylacetamide;
(R)-3-((8-chloro-1-(2,6-dichloro-4-(2-hydroxypropoxy)phenyl)-2-methyl-4-oxo-1,4-dihydro-1,6-naphthyridin-5-yl)oxy)-N-methylpropanamide;
(R)-3-((8-chloro-1-(2,6-dichloro-4-(2-hydroxyethoxy)phenyl)-2-methyl-4-oxo-1,4-dihydro-1,6-naphthyridin-5-yl)oxy)-N, 2-dimethylpropanamide;
(S)-3-((8-chloro-1-(2,6-dichloro-4-(2-hydroxyethoxy)phenyl)-2-methyl-4-oxo-1,4-dihydro-1,6-naphthyridin-5-yl)oxy)-N,2-dimethylpropanamide;
N-(2-((8-chloro-1-(2,6-dichloro-4-(2-hydroxyethoxy)phenyl)-2-methyl-4-oxo-1,4-dihydro-1,6-naphthyridin-5-yl)oxy)ethyl)acetamide;
(S)-3-((8-chloro-1-(2,6-dichloro-4-(2-hydroxypropoxy)phenyl)-2-methyl-4-oxo-1,4-dihydro-1,6-naphthyridin-5-yl)oxy)-N-methylpropanamide;
3-((8-chloro-1-(2,6-dichloro-4-(2-hydroxyethoxy)phenyl)-2-methyl-4-oxo-1,4-dihydro-1,6-naphthyridin-5-yl)oxy)-N,2,2-trimethylpropanamide;
(R)-3-((8-chloro-1-(2,6-dichloro-4-(2-hydroxyethoxy)phenyl)-2-methyl-4-oxo-1,4-dihydro-1,6-naphthyridin-5-yl)oxy)-2-hydroxy-N-methylpropanamide;
(S)-3-((8-chloro-1-(2,6-dichloro-4-(2-hydroxyethoxy)phenyl)-2-methyl-4-oxo-1,4-dihydro-1,6-naphthyridin-5-yl)oxy)-2-hydroxy-N-methylpropanamide;
(S)-3-((8-chloro-1-(2,6-dichloro-4-(2-hydroxy-2-methylpropoxy)phenyl)-2-methyl-4-oxo-1,4-dihydro-1,6-naphthyridin-5-yl)oxy)-2-hydroxy-N-methylpropanamide;
3-((8-chloro-1-(2,6-dichloro-4-(2-hydroxyethoxy)phenyl)-2-methyl-4-oxo-1,4-dihydro-1,6-naphthyridin-5-yl)oxy)-2,2-difluoro-N-methylpropanamide;
3-((8-chloro-1-(2,6-dichloro-4-(2-hydroxyethoxy)phenyl)-2-methyl-4-oxo-1,4-dihydro-1,6-naphthyridin-5-yl)oxy)propanamide;
N-(3-(8-chloro-1-(2,6-dichloro-4-(2-hydroxyethoxy)phenyl)-2-methyl-4-oxo-1,4-dihydro-1,6-naphthyridin-5-yl)propyl)acctamide;
8-chloro-1-(2,6-dichloro-4-(2-hydroxyethoxy)phenyl)-2-methyl-5-(2-(methylsulfonyl)ethyl)-1,6-naphthyridin-4(1H)-one;
8-chloro-1-(2,6-dichloro-4-(2-hydroxyethoxy)phenyl)-2-methyl-5-(2-(methylsulfonyl)propyl)-1,6-naphthyridin-4(1H)-one; and
8-chloro-1-(2,6-dichloro-4-(2-hydroxy-2-methylpropoxy)phenyl)-2-methyl-5-(3-(methylsulfonyl)propyl)-1,6-naphthyridin-4(1H)-one.

8. A compound according to claim 1 or a pharmaceutically acceptable salt thereof, said compound being selected from the group consisting of:
2-(3,5-dichloro-4-(8-chloro-5-(2-hydroxy-3-(methylamino)-3-oxopropoxy)-2-methyl-4-oxo-1,6-naphthyridin-1(4H)-yl)phenoxy)ethyl dihydrogen phosphate;
(R)-2-(3,5-dichloro-4-(8-chloro-5-(2-hydroxy-3-(methylamino)-3-oxopropoxy)-2-methyl-4-oxo-1,6-naphthyridin-1(4H)-yl)phenoxy)ethyl dihydrogen phosphate; and
(S)-2-(3,5-dichloro-4-(8-chloro-5-(2-hydroxy-3-(methylamino)-3-oxopropoxy)-2-methyl-4-oxo-1,6-naphthyridin-1(4H)-yl)phenoxy)ethyl dihydrogen phosphate.

9. A compound according to claim 1 or a pharmaceutically acceptable salt thereof, said compound being selected from the group consisting of:
3-((8-chloro-1-(2,6-dichloro-4-(2-hydroxyethoxy)phenyl)-2-methyl-4-oxo-1,4-dihydro-1,6-naphthyridin-5-yl)oxy)-1-(methylamino)-1-oxopropan-2-yl dihydrogen phosphate;
(R)-3-((8-chloro-1-(2,6-dichloro-4-(2-hydroxyethoxy)phenyl)-2-methyl-4-oxo-1,4-dihydro-1,6-naphthyridin-5-yl)oxy)-1-(methylamino)-1-oxopropan-2-yl dihydrogen phosphate; and
(S)-3-((8-chloro-1-(2,6-dichloro-4-(2-hydroxyethoxy)phenyl)-2-methyl-4-oxo-1,4-dihydro-1,6-naphthyridin-5-yl)oxy)-1-(methylamino)-1-oxopropan-2-yl dihydrogen phosphate.

10. A pharmaceutical composition comprising a compound according to claim 1 or a pharmaceutically acceptable salt thereof and one or more pharmaceutically acceptable carriers.

11. The pharmaceutical composition according to claim 10. further comprising at least one additional pharmaceutically active agent selected from Class I antiarrhythmic agents. Class II antiarrhythmic agents, Class III antiarrhythmic agents, Class IV antiarrhythmic agents, Class V antiarrhythmic agents, cardiac glycosides and other drugs affecting atrial refractoriness; haemostasis modulators, antithrombotics, thrombin inhibitor;: factor VIla inhibitors; anticoagulants, factor Xa inhibitors, and direct thrombin inhibitors; antiplatelet agents, cyclooxygenase inhibitors, adenosine diphosphate (ADP) receptor inhibitors, phosphodiesterase inhibitors, glycoprotein IIB/IIA, adenosine reuptake inhibitors; anti-dyslipidemia agents, HMG-CoA reductase inhibitors, other cholesterol-lowering agents; bile acid sequestrants; cholesterol absorption inhibitors; cholesteryl ester transfer protein (CETP) inhibitors; inhibitors of the ileal bile acid transport system (IBAT inhibitors); bile acid binding resins; nicotinic acid and analogues thereof; anti-oxidants; omega-3 fatty acids; antihypertensive agents, including adrenergic receptor antagonists, beta blockers, alpha blockers, mixed alpha/beta blockers; adrenergic receptor agonists, alpha-2 agonists; angiotensin converting enzyme (ACE) inhibitors, calcium channel blockers; angiotensin II receptor antagonists; aldosterone receptor antagonists; centrally acting adrenergic drugs, central alpha agonists; and diuretic agents; anti-obesity agents, pancreatic lipase inhibitors, microsomal transfer protein (MTP) modulators, diacyl glycerolacyltransferase (DGAT) inhibitors, cannabinoid (CBI) receptor antagonists; insulin and insulin analogues; insulin secretagogues; agents that improve incretin action, dipeptidyl peptidase IV (DPP-4) inhibitors, glucagon-like peptide-I (GLP-1) agonists; insulin sensitizing agents, peroxisome proliferator activated receptor gamma (PPARγ) agonists, agents that modulate hepatic glucose balance, fructose 1,6-bisphosphatase inhibitors, glycogen phosphorylase inhibitors, glycogen synthase kinase inhibitors, glucokinase activators; agents designed to reduce/slow the absorption of glucose from the intestine, alpha-glucosidase inhibitors; agents which antagonize the actions of or reduce secretion of glucagon, amylin analogues; agents that prevent the reabsorption of glucose by the kidney, and sodium-dependent glucose transporter 2 (SGLT-2) inhibitors.

12. A method for treating or preventing a disease or disorder responsive to the inhibition of the GIRK1/4 receptor, wherein the disease or disorder responsive to the inhibition of the GIRK1/4 receptor is selected from cardiac arrhythmia, atrial fibrillation, bradyarrhythmia. bradycardia, heart block, sick sinus syndrome, parasympathetic hyperactivation, primary hyperaldosteronism, hypotension, and vasovagal syncope, the method comprising administering to a patient in need thereof a therapeutically effective amount of a compound according to claim 1, or a pharmaceutically acceptable salt thereof.

13. The method according to claim 12, wherein administering the compound is orally.

14. A method for treating or preventing a disease or disorder responsive to the inhibition of the GIRK1/4 receptor, wherein the disease or disorder responsive to the inhibition of the GIRK1/4 receptor is selected from cardiac arrhythmia, atrial fibrillation, bradyarrhythmia, bradycardia, heart block, sick sinus syndrome, parasympathetic hyperactivation, primary hyperaldosteronism, hypotension, and vasovagal syncope, the method comprising administering to a patient in need thereof a therapeutically effective amount of a pharmaceutical composition according to claim 10.

15. The method according to claim 14, wherein administering the pharmaceutical composition is orally.

16. A method for maintaining a sinus rhythm after cardioversion in a patient with persistent or recent onset of atrial fibrillation or preventing a recurrence in a patient with paroxysmal atrial fibrillation, the method comprising administering to a patient in need thereof a therapeutically effective amount of a compound according to claim 1 or a pharmaceutically acceptable salt thereof.

17. The method according to claim 16, wherein administering the compound is orally.

18. A method for maintaining a sinus rhythm after cardioversion in a patient with persistent or recent onset of atrial fibrillation or preventing a recurrence in a patient with paroxysmal atrial fibrillation, the method comprising administering to a patient in need thereof a therapeutically effective amount of a pharmaceutical composition according to claim 10.

19. The method according to claim 18, wherein administering the pharmaceutical composition is orally.
